(19) United States
(12) Patent Application Publication
(10) Pub. No.: US 2024/0350423 A1
(43) Pub. Date: Oct. 24, 2024

(54) NANOFIBER SHEET FOR HEALING TEAR OF ROTATOR CUFF CONTAINING RECOMBINANT PARATHYROID HORMONE AND METHOD FOR MANUFACTURING THE SAME

(71) Applicants: Seoul National University Hospital, Seoul (KR); Pusan National University Industry-University Cooperation Foundation, Busan (KR)

(72) Inventors: Joo Han Oh, Seongnam-Si (KR); Hyeong Jang Jeong, Seongnam-Si (KR); Suk-Hee Park, Busan (KR)

(21) Appl. No.: 18/224,561

(22) Filed: Jul. 20, 2023

(30) Foreign Application Priority Data
Jul. 20, 2022 (KR) 10-2022-0089370
Mar. 15, 2023 (KR) 10-2023-0033738

(51) Int. Cl. A61K 9/70; A61K 9/00; A61K 38/29; A61K 47/36; A61P 19/02

(52) U.S. Cl. CPC A61K 9/7007; A61K 9/0024; A61K 38/29; A61K 47/36; A61P 19/02

(57) ABSTRACT

Provided is a nanofiber sheet for healing after rotator cuff repair containing recombinant parathyroid hormone and a method of manufacturing the same. The nanofiber sheet including a composition containing recombinant parathyroid hormone therein does not cause complications, and has an excellent effect of healing of rotator cuff tears by improving tendon to bone healing when the sheet is locally fixed or attached to a suture site, specifically rotator cuff tear site or rotator cuff suture site, compared to direct systemic or local administration of recombinant parathyroid hormone.

### CROSS-REFERENCE TO RELATED APPLICATION

This application is based on and claims priority to Korean Patent Application No. 10-2022-0089370 filed on Jul. 20, 2022 and Korean Patent Application No. 10-2023-0033738 filed on Mar. 15, 2023, in the Korean Intellectual Property Office, the disclosures of which are incorporated by reference herein in their entirety.

### INCORPORATION BY REFERENCE STATEMENT REGARDING THE MATERIAL IN THE SEQUENCE LISTING XML FILE

The XML file submitted herewith is incorporated by reference in the Specification. The XML file is identified as follows: (i) Name of the File: OF23P089US_sequence list; (ii) Date of Creation: Jun. 10, 2024; (iii) Size of File: 13,504 bytes.

### BACKGROUND

### 1. Field

Disclosed herein is a nanofiber sheet for healing of rotator cuff tears containing recombinant parathyroid hormone and a method for manufacturing the same.

### 2. Description of the Related Art

Rotator cuff tears (RCTs) are a common and progressive disease of the upper limbs that causes pain and disability of the shoulder joint. Fortunately, with the rapid development of surgical technology, satisfactory treatment results have been obtained [Carbonel I, Martinez A A, Aldea E, Ripalda J, Herrera A. Outcome and structural integrity of rotator cuff after arthroscopic treatment of large and massive tears with double row technique: a 2-year followup. Adv Orthop 2013; 2013:914148. doi:10.1155/2013/914148; Melillo A S, Savoie F H, 3rd, Field L D. Massive rotator cuff tears: debridement versus repair. Orthop Clin North Am 1997; 28(1):117-24]. However, despite advances in surgical technology, postoperative healing failure remains a frequent complication with failure rates ranging from 11 to 94% [Galatz L M, Ball C M, Teefey S A, Middleton W D, Yamaguchi K. The outcome and repair integrity of completely arthroscopically repaired large and massive rotator cuff tears. J Bone Joint Surg Am 2004; 86(2):219-24. doi:10.2106/00004623-200402000-00002; Le B T, Wu X L, Lam P H, Murrell G A. Factors predicting rotator cuff retears: an analysis of 1000 consecutive rotator cuff repairs. Am J Sports Med 2014; 42(5):1134-42. doi:10.1177/0363546514525336].

Numerous risk factors have been suspected to contribute to the healing failure [Cho N S, Rhee Y G. The factors affecting the clinical outcome and integrity of arthroscopically repaired rotator cuff tears of the shoulder. Clin Orthop Surg 2009; 1(2):96-104. doi:10.4055/cios.2009.1.2.96]. In addition, osteoporosis is an independent prognostic factor for rotator cuff healing [Chung S W, Oh J H, Gong H S, Kim J Y, Kim S H. Factors affecting rotator cuff healing after arthroscopic repair: osteoporosis as one of the independent risk factors. Am J Sports Med 2011; 39(10):2099-107. doi:10.1177/0363546511415659]. The recombinant human parathyroid hormone (rhPTH) has been reported to significantly improve bone mineral density and rotator cuff healing [Compston J E. Skeletal actions of intermittent parathyroid hormone: effects on bone remodelling and structure. Bone 2007; 40(6):1447-52. doi:10.1016/j.bone.2006.09.008; Hettrich C M, Beamer B S, Bedi A, Deland K, Deng X H, Ying L, et al. The effect of rhPTH on the healing of tendon to bone in a rat model. J Orthop Res 2012; 30(5):769-74. doi:10.1002/jor.22006]. Since rhPTH can regulate calcium homeostasis by preventing the loss of calcium ions in vivo [Andreassen T T, Ejersted C, Oxlund H. Intermittent parathyroid hormone (1-34) treatment increases callus formation and mechanical strength of healing rat fractures. J Bone Miner Res 1999; 14(6):960-8; Dempster D W, Cosman F, Parisien M, Shen V, Lindsay R. Anabolic actions of parathyroid hormone on bone. Endocr Rev 1993; 14(6):690-709; Murray T M, Rao L G, Divieti P, Bringhurst F R. Parathyroid hormone secretion and action: evidence for discrete receptors for the carboxyl-terminal region and related biological actions of carboxyl-terminal ligands. Endocr Rev 2005; 26(1):78-113. Doi:10.1210/er.2003-0024], there is growing interest in biologics that promote rotator cuff healing [Chen X, Giambini H, Ben-Abraham E, An K N, Nassr A, Zhao C. Effect of Bone Mineral Density on Rotator Cuff Tear: An Osteoporotic Rabbit Model. PLoS One 2015; 10(10):e0139384. doi:10.1371/journal.pone.0139384; Duchman K R, Goetz J E, Uribe B U, Amendola A M, Barber J A, Malandra A E, et al. Delayed administration of recombinant human parathyroid hormone improves early biomechanical strength in a rat rotator cuff repair model. J Shoulder Elbow Surg 2016; 25(8):1280-7. doi:10.1016/j.jse.2015.12.016; Hettrich C M, Beamer B S, Bedi A, Deland K, Deng X H, Ying L, et al. The effect of rhPTH on the healing of tendon to bone in a rat model. J Orthop Res 2012; 30(5):769-74. doi:10.1002/jor.22006; Yoon J P, Chung S W, Jung J W, Lee Y S, Kim K I, Park G Y, et al. Is a Local Administration of Parathyroid Hormone Effective to Tendon-to-Bone Healing in a Rat Rotator Cuff Repair Model? J Orthop Res 2020; 38(1):82-91. doi:10.1002/jor.24452].

It is important to restore the tendon to bone junction after rotator cuff repair. The native rotator cuff insertion on the humeral head consists of four typical areas: tendon, non-mineralized fibrocartilage, mineralized fibrocartilage, and bone. Precisely, rhPTH positively affects the reconstruction of the fibrocartilage area through the chondrogenic pathway [Kakar S, Einhorn T A, Vora S, Miara L J, Hon G, Wigner N A, et al. Enhanced chondrogenesis and Wnt signaling in PTH-treated fractures. J Bone Miner Res 2007; 22(12):1903-12. doi:10.1359/jbmr.070724; Nakazawa T, Nakajima A, Shiomi K, Moriya H, Einhorn T A, Yamazaki M. Effects of low-dose, intermittent treatment with recombinant human parathyroid hormone (1-34) on chondrogenesis in a model of experimental fracture healing. Bone 2005; 37(5):711-9. doi:10.1016/j.bone.2005.06.013]. The formation of fibrocartilage and the generation of type 1 procollagen-producing cells within the rotator cuff insertion may be significantly enhanced following daily systemic rhPTH administration in rats after rotator cuff repair [Hettrich C M, Beamer B S, Bedi A, Deland K, Deng X H, Ying L, et al. The effect of rhPTH on the healing of tendon to bone in a rat model. J Orthop Res 2012; 30(5):769-74. doi:10.1002/jor.22006]. A clinical study reported that rhPTH could be a systemic treatment option for significantly improving tendon to bone healing after arthroscopic rotator cuff repair in patients with RCT greater than 2 cm [Oh J H, Kim D H, Jeong H J, Park J H, Rhee S M. Effect of Recombinant Human Parathyroid Hormone on Rotator Cuff Healing After Arthroscopic Repair. Arthroscopy 2019; 35(4):1064-71. doi:10.1016/j.arthro.2018.11.038].

However, the systemic rhPTH treatment may cause several side effects [Luigetti M, Capone F, Monforte M, Di Lazzaro V. Muscle cramps and weakness after teriparatide therapy: a new drug-induced myopathy? Muscle Nerve 2013; 47(4):615. doi:10.1002/mus.23661; Migliaccio S, Resmini G, Buffa A, Fornari R, Di Pietro G, Cerocchi I, et al. Evaluation of persistence and adherence to teriparatide treatment in patients affected by severe osteoporosis (PATT): a multicenter observational real life study. Clin Cases Miner Bone Metab 2013; 10(1):56-60. doi:10.11138/ccmbm/2013.10.1.056; Thiruchelvam N, Randhawa J, Sadiek H, Kistangari G. Teriparatide induced delayed persistent hypercalcemia. Case Rep Endocrinol 2014; 2014:802473. doi:10.1155/2014/802473]. Therefore, topical rhPTH administration has generated considerable interest for many researchers evaluating effectiveness in animal models [Auersvald C M, Santos F R, Nakano M M, Leoni G B, de Sousa Neto M D, Scariot R, et al. The local administration of parathyroid hormone encourages the healing of bone defects in the rat calvaria: Micro-computed tomography, histological and histomorphometric evaluation. Arch Oral Biol 2017; 79:14-9. doi:10.1016/j.archoralbio.2017.02.016; Dang M, Koh A J, Jin X, McCauley L K, Ma P X. Local pulsatile PTH delivery regenerates bone defects via enhanced bone remodeling in a cell-free scaffold. Biomaterials 2017; 114:1-9. doi:10.1016/j.biomaterials.2016.10.049; Tokunaga K, Seto H, Ohba H, Mihara C, Hama H, Horibe M, et al. Topical and intermittent application of parathyroid hormone recovers alveolar bone loss in rat experimental periodontitis. J Periodontal Res 2011; 46(6):655-62. doi:10.1111/j.1600-0765.2011.01386.x]. However, studies of topical rhPTH administration are mostly limited to bone formation and bone mineral density [Auersvald C M, Santos F R, Nakano M M, Leoni G B, de Sousa Neto M D, Scariot R, et al. The local administration of parathyroid hormone encourages the healing of bone defects in the rat calvaria: Micro-computed tomography, histological and histomorphometric evaluation. Arch Oral Biol 2017; 79:14-9. doi:10.1016/j.archoralbio.2017.02.016], and effects on rotator cuff tendon to bone healing have been rarely reported. As for the topical administration route, direct injection using syringes, alginate scaffolds, and collagen sponges have been reported [Auersvald C M, Santos F R, Nakano M M, Leoni G B, de Sousa Neto M D, Scariot R, et al. The local administration of parathyroid hormone encourages the healing of bone defects in the rat calvaria: Micro-computed tomography, histological and histomorphometric evaluation. Arch Oral Biol 2017; 79:14-9. doi:10.1016/j.archoralbio.2017.02.016; Yoon J P, Chung S W, Jung J W, Lee Y S, Kim K I, Park G Y, et al. Is a Local Administration of Parathyroid Hormone Effective to Tendon-to-Bone Healing in a Rat Rotator Cuff Repair Model? J Orthop Res 2020; 38(1):82-91. doi:10.1002/jor.24452]. Nevertheless, the studies of the topical rhPTH administration method for rotator cuff healing are still insufficient, and the results are controversial. Nanofiber scaffolds are a recent discovery in the field of biological materials as a topical administration tool because they can be used for certain drug-delivery applications and provide an extracellular matrix (ECM) environment necessary for tendon healing [Riggin C N, Qu F, Kim D H, Huegel J, Steinberg D R, Kuntz A F, et al. Electrospun PLGA Nanofiber Scaffolds Release Ibuprofen Faster and Degrade Slower After In Vivo Implantation. Ann Biomed Eng 2017; 45(10):2348-59. doi:10.1007/s10439-017-1876-7; Wang X, Ding B, Li B. Biomimetic electrospun nanofibrous structures for tissue engineering. Mater Today (Kidlington) 2013; 16(6):229-41. doi:10.1016/j.mattod.2013.06.005]. In addition, the nanofiber organization and alignment tailored to the functional needs of the rotator cuff tendon may be adjusted during fabrication [Li W J, Mauck R L, Cooper J A, Yuan X, Tuan R S. Engineering controllable anisotropy in electrospun biodegradable nanofibrous scaffolds for musculoskeletal tissue engineering. J Biomech 2007; 40(8):1686-93. doi:10.1016/j.jbiomech.2006.09.004; Ma Z, Kotaki M, Inai R, Ramakrishna S. Potential of nanofiber matrix as tissue-engineering scaffolds. Tissue Eng 2005; 11(1-2):101-9. doi:10.1089/ten.2005.11.101].

Accordingly, this study was conducted to verify the effect of topical rhPTH administration using a biomimetic nanofiber sheet for rotator cuff healing.

### SUMMARY

This study was conducted, based on the hypothesis that the 3D printed, rhPTH-containing nanofiber sheet would support and improve rotator cuff healing compared to direct topical rhPTH administration, to confirm its effect in a rabbit chronic RCT model. As a result, this study has confirmed that the nanofiber sheet including the rhPTH-containing composition has an effect of healing of rotator cuff tears.

Accordingly, in an aspect, an object of the present invention is to provide a nanofiber sheet capable of healing of rotator cuff tears and a manufacturing method thereof.

In an aspect, the present invention provides a nanofiber sheet for healing of rotator cuff tears including a composition containing teriparatide, isomers thereof, pharmaceutically acceptable salts thereof, hydrates thereof, or solvates thereof as an active ingredient.

In another aspect, the present invention provides a method of manufacturing the nanofiber sheet for healing of rotator cuff tears, the method comprising incorporating a composition containing teriparatide, isomers thereof, pharmaceutically acceptable salts thereof, hydrates thereof, or solvates thereof as an active ingredient in a nanofiber sheet.

The nanofiber sheet according to an aspect of the present invention including a composition containing recombinant parathyroid hormone therein does not cause complications, and has an excellent effect of healing of rotator cuff tears by improving tendon to bone healing when the sheet is locally fixed or attached to a suture site, specifically rotator cuff tear site or rotator cuff suture site, compared to direct systemic or local administration of recombinant parathyroid hormone.

### BRIEF DESCRIPTION OF THE DRAWINGS

The above and other aspects, features and advantages of the disclosed example embodiments will be more apparent from the following detailed description taken in conjunction with the accompanying drawings in which:

FIG. 1A is a scanning electron microscope image of polycaprolactone nanofibers, and FIG. 1B is a is an image illustrating the final shape and size of a three-dimensionally (3D) printed nanofiber sheet according to an embodiment of the present invention;

FIG. 2 is an image illustrating the final form of a 3D printed, recombinant parathyroid hormone-soaked nanofiber sheet according to an embodiment of the present invention;

FIG. 3 is a flowchart of a study design according to an embodiment of the present invention, wherein the abbreviations in FIG. 3 are as follows: HA (hyaluronic acid), rhPTH (recombinant human parathyroid hormone);

FIG. 4 is a fixed image of a 3D printed, parathyroid hormone-soaked nanofiber sheet on a tendon to bone junction when a torn supraspinatus tendon is sutured (treated) according to an embodiment of the present invention;

FIG. 5A is an image of testing parameters of a biomechanical evaluation, using a universal material testing machine and a custom fixture clamping system, according to an embodiment of the present invention and FIG. 5B is an image of a state in which a humeral head is firmly fixed to a humeral head fixing part and a supraspinatus tendon protrudes through a hole;

FIG. 6A is representative photomicrographs (40× magnification) of tendon to bone junctions stained with hematoxylin and eosin after 4 weeks and 12 weeks of treatment, respectively, according to an embodiment of the present invention, and FIG. 6B is representative photomicrographs (40× magnification) of tendon to bone junctions stained with Masson's tricolor after 4 weeks and 12 weeks of treatment, respectively;

FIG. 7 is a graph showing load-to-failure results of tissues treated in a biomechanical evaluation according to an embodiment of the present invention, wherein group E showed significantly higher load-to-failure than the other groups (P<0.001) in FIG. 7;

FIG. 8A is a schematic diagram for illustrating a method of manufacturing a rhPTH biocomposite, and FIGS. 8B to 8D are images of the manufactured nanofiber sheet;

FIG. 9 is a flowchart of the experimental design of Example 2, which is an embodiment of the present invention; and

FIGS. 10A to 10C are fixed images of a 3D printed rhPTH-soaked nanofiber sheet and a rhPTH biocomposite on a tendon to bone junction when a torn proximal humerus is sutured (treated) according to an embodiment of the present invention.

### DETAILED DESCRIPTION

Hereinafter, embodiments of the present invention will be described in more detail.

The embodiments of the present invention may be embodied in various forms and shall not be construed as being limited to embodiments described herein. In the description, details of features and techniques may be omitted to more clearly disclose example embodiments.

Accordingly, such improvements and modifications will fall within the scope of protection of the present invention as long as it is obvious to those skilled in the art.

The terminology used herein is for the purpose of describing particular embodiments only and is not intended to be limiting. As used herein, the singular forms “a,” “an” and “the” are intended to include the plural forms as well, unless the context clearly indicates otherwise. Furthermore, the use of the terms a, an, etc. do not denote a limitation of quantity, but rather denote the presence of at least one of the referenced items.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein. All methods described herein can be performed in a suitable order unless otherwise indicated herein or otherwise clearly contradicted by context. The use of any and all examples, or exemplary language (e.g., “such as”), is intended merely to better illustrate the example embodiments and does not pose a limitation on the scope of the present disclosure unless otherwise claimed. No language in the specification should be construed as indicating any non-claimed element as essential to the practice of the present disclosure as used herein.

In this application, it is to be understood that the terms “include” or “have” are intended to indicate there is a feature, a number, a step, an operation, a component, a part, or a combination thereof described in the specification, and not to exclude the presence or addition of one or more other features, numbers, steps, operations, components, parts, or combinations thereof.

In an aspect of the present invention, a nanofiber sheet for healing of rotator cuff tears is provided, wherein the nanofiber sheet includes a composition containing teriparatide, isomers thereof, pharmaceutically acceptable salts thereof, hydrates thereof, or solvates thereof as an active ingredient.

The teriparatide according to an aspect of the present invention is recombinant human parathyroid hormone (rhPTH) and may have the structure of Formula 1 below. The teriparatide is the same as part of human parathyroid hormone (PTH), and the intermittent use of the teriparatide stimulates bone formation by activating osteoblasts rather than osteoclasts, thereby improving bone mineral density (BMD) as a result.

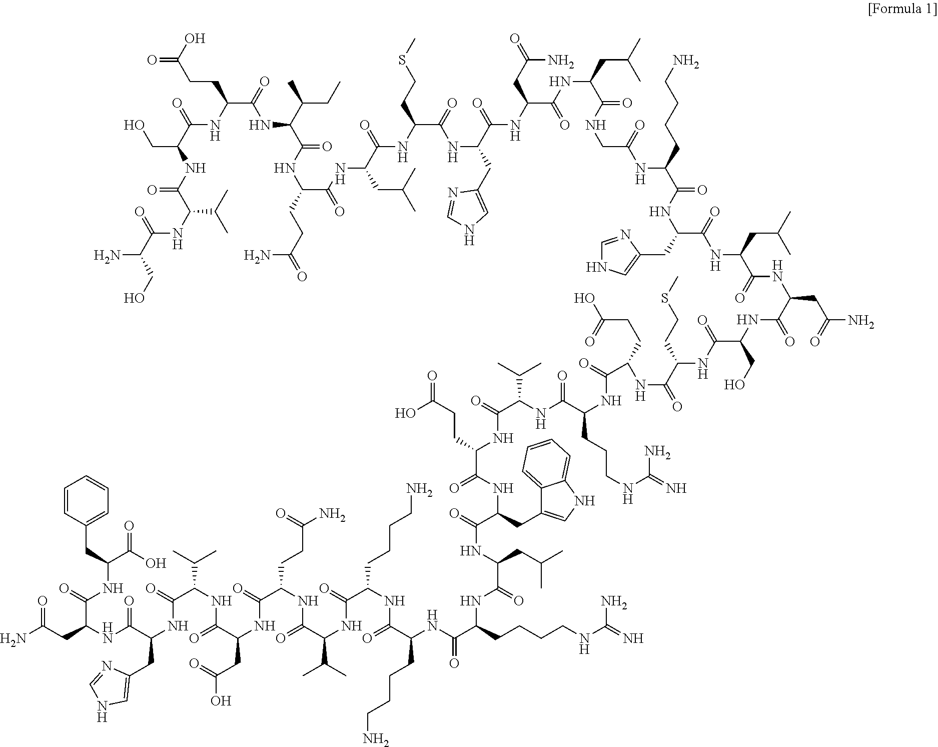

The phrase “pharmaceutically acceptable” according to an aspect of the present invention indicates that it is recognized as being approved or capable of being approved by a governmental or equivalent regulatory body for use in animals, specifically in humans, or being listed in a pharmacopeia or other general pharmacopoeia by avoiding significant toxic effects when used in normal medicinal dosage.

The phrase “pharmaceutically acceptable salts” according to an aspect of the present invention refers to salts that are pharmaceutically acceptable and have the desired pharmacological activity of the parent compound, according to an aspect of the present invention. The salts may include (1) acid addition salts formed with inorganic acids such as hydrochloric acid, hydrobromic acid, sulfuric acid, nitric acid, phosphoric acid and the like, or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfate, gluconic acid, glutamic acid, hydroxynaphthoic acid, salicylic acid, stearic acid, muconic acid; or (2) salts formed when an acidic proton present in the parent compound is substituted.

The term “isomers” according to an aspect of the present invention includes not only optical isomers (e.g., essentially pure enantiomers, essentially pure diastereomers or a mixture thereof), but also conformational isomers (i.e., isomers that differ only in the angle of one or more chemical bonds), position isomers (particularly tautomers) or geometric isomers (e.g., cis-trans isomers).

The term “hydrates” according to an aspect of the present invention refers to compounds to which water is bonded, and broadly, includes inclusion compounds that do not have a chemical bond between water and the compound.

The term “solvates” according to an aspect of the present invention refers to higher order compounds formed between solute molecules or ions and solvent molecules or ions.

The composition according to an aspect of the present invention may further include hyaluronic acid or salts thereof. The hyaluronic acid or salts thereof may be a carrier of teriparatide, isomers thereof, pharmaceutically acceptable salts thereof, hydrates thereof, or solvates thereof. Although hyaluronic acid and nanofiber scaffolds have been reported to support rotator cuff healing in previous studies [Honda H, Gotoh M, Kanazawa T, Ohzono H, Nakamura H, Ohta K, et al. Hyaluronic Acid Accelerates Tendon-to-Bone Healing After Rotator Cuff Repair. Am J Sports Med 2017; 45(14):3322-30. doi:10.1177/0363546517720199; Moffat K L, Kwei A S, Spalazzi J P, Doty S B, Levine W N, Lu H H. Novel nanofiber-based scaffold for rotator cuff repair and augmentation. Tissue Eng Part A 2009; 15(1):115-26. Doi:10.1089/ten.tea.2008.0014], hyaluronic acid and nanofiber scaffolds did not have a significant effect on tendon to bone healing in the previous studies, and the nanofiber scaffolds are known to rapidly burst release the agent with which the scaffolds are impregnated at the initial stage of implantation (attachment or fixation to the inside of the body). However, the nanofiber sheet according to an embodiment of the present invention further includes hyaluronic acid or salts thereof in the composition, and the hyaluronic acid or salts thereof as a teriparatide carrier is capable of controlling the concentration of teriparatide, isomers thereof, pharmaceutically acceptable salts thereof, hydrates thereof, or solvates thereof in the nanofiber sheet for a long time, and thus plays important role [Gentile P, Nandagiri V K, Pabari R, Daly J, Tonda-Turo C, Ciardelli G, et al. Influence of Parathyroid Hormone-Loaded PLGA Nanoparticles in Porous Scaffolds for Bone Regeneration. Int J Mol Sci 2015; 16(9):20492-510. doi:10.3390/ijms160920492].

The nanofiber sheet, according to an aspect of the present invention, may be impregnated with a composition containing teriparatide, isomers thereof, pharmaceutically acceptable salts thereof, hydrates thereof, or solvates thereof as an active ingredient.

In the nanofiber sheet according to an aspect of the present invention, the nanofibers constituting the nanofiber sheet may contain a composition therein including teriparatide, isomers thereof, pharmaceutically acceptable salts thereof, hydrates thereof, or solvates thereof as an active ingredient.

In the nanofiber sheet according to an aspect of the present invention, the nanofibers may have a core-shell structure in which a nanofiber shell surrounds the core made of a composition including teriparatide, isomers thereof, pharmaceutically acceptable salts thereof, hydrates thereof, or solvates thereof as an active ingredient.

The nanofiber sheet, according to an aspect of the present invention, may be used for wound healing, wherein the wound may be one selected from the group consisting of non-healing traumatic wound, destruction of tissue by irradiation, abrasion, laceration, avulsion, penetrating wound, gunshot wound, cut, burn, frostbite, skin ulcer, skin dryness, keratosis, crack, burst, dermatitis, surgical or vascular disease wound, bruise, corneal wound, decubitus ulcer, bedsore, chronic ulcer, postoperative suture, spinal injury, gynecological wound, chemical wound, and acne, specifically a wound at the suture site after rotator cuff surgery, more specifically a wound at the suture site after rotator cuff repair, more specifically a tear at the suture site after rotator cuff repair, and more specifically a tear or rupture at the suture site in a size greater than 2 cm after rotator cuff repair.

The rotator cuff tear according to an aspect of the present invention may be a chronic or degenerative rotator cuff tear.

The healing of rotator cuff tear according to an aspect of the present invention may be healing of a tear at a suture site after rotator cuff repair, and more specifically, healing of a tear or rupture at a suture site in a size greater than 2 cm after rotator cuff repair, but is not limited thereto.

The tear refers to split, burst, rip, or crack, and the tear may specifically include split, burst, rip, or crack at a suture site after rotator cuff repair, but is not limited thereto.

The healing of rotator cuff tear according to an aspect of the present invention may reduce re-tear after rotator cuff repair.

The nanofiber sheet, according to an aspect of the present invention, may be attached or fixed for 1 to 48 weeks after rotator cuff repair, specifically, for 1 week to 12 months. Specifically, a period of attaching or fixing the composition may be 1 week or more, 2 weeks or more, 3 weeks or more, 4 weeks or more, 5 weeks or more, 6 weeks or more, 7 weeks or more, 8 weeks or more, 9 weeks or more, 10 weeks or more, 11 weeks or more, 12 weeks or more, 13 weeks or more, 14 weeks or more, 15 weeks or more, 16 weeks or more, 17 weeks or more, 18 weeks or more, 19 weeks or more, 20 weeks or more, 21 weeks or more, 22 weeks or more, 23 weeks or more, 24 weeks or more, 25 weeks or more, 26 weeks or more, 27 weeks or more, 28 weeks or more, 29 weeks or more, 30 weeks or more, 31 weeks or more, 32 weeks or more, 33 weeks or more, 34 weeks or more, 35 weeks or more, 36 weeks or more, 37 weeks or more, 38 weeks or more, 39 weeks or more, 40 weeks or more, 41 weeks or more, 42 weeks or more, 43 weeks or more, 44 weeks or more, 45 weeks or more, 46 weeks or more, or 47 weeks or more, and 48 weeks or less, 47 weeks or less, 46 weeks or less, 45 weeks or less, 44 weeks or less, 43 weeks or less, 42 weeks or less, 41 weeks or less, 40 weeks or less, 39 weeks or less, 38 weeks or less, 37 weeks or less, 36 weeks or less 35 weeks or less, 34 weeks or less, 33 weeks or less, 32 weeks or less, 31 weeks or less, 30 weeks or less, 29 weeks or less, 28 weeks or less, 27 weeks or less, 26 weeks or less, 25 weeks or less, 24 weeks or less, 23 weeks or less, 22 weeks or less, 21 weeks or less, 20 weeks or less, 19 weeks or less, 18 weeks or less, 17 weeks or less, 16 weeks or less, 15 weeks or less, 14 weeks or less, 13 weeks or less, 12 weeks or less, 11 weeks or less 10 weeks or less, 9 weeks or less, 8 weeks or less, 7 weeks or less, 6 weeks or less, 5 weeks or less, 4 weeks or less, 3 weeks or less, or 2 weeks or less after rotator cuff repair. Alternatively, the period of attaching or fixing the composition may be 1 week or more, 2 weeks or more, 3 weeks or more, 4 weeks or more, 1 month or more, 2 months or more, 3 months or more, 4 months or more, 5 months or more, 6 months or more, 7 months or more, 8 months or more, 9 months or more, 10 months or more, 11 months or more, and 12 months or less, 11 months or less, 10 months or less, 9 months or less, 8 months or less, 7 months or less, 6 months or less, 5 months or less, 4 months or less, 3 months or less, 2 months or less, 1 month or less after rotator cuff repair. The teriparatide, the active ingredient of the composition, may have side effects such as nausea, vomiting, itching, and muscle spasms, and the longer the administration period of teriparatide, the higher the possibility of occurrence of the side effects. Therefore, the period of attaching or fixing the composition containing teriparatide as an active ingredient may vary depending on the age, sex, weight of the subject to be treated, the specific disease or pathological condition to be treated, the severity of the disease or pathological condition, the route of administration, and the judgment of the prescriber, and the period of attaching or fixing the composition is determined based on these factors within the level of those skilled in the art, but is not limited thereto.

The nanofiber sheet, according to an aspect of the present invention, may be biodegradable.

The nanofiber sheet, according to an aspect of the present invention, may be attached or fixed after rotator cuff repair. Specifically, the nanofiber sheet may be attached or fixed within 1 second, within 10 seconds, within 1 minute, within 2 minutes, within 5 minutes, within 10 minutes, within 1 hour, within 2 hours, within 4 hours, within 6 hours, within 12 hours, within 24 hours, within 2 days, within 3 days, within 4 days, within 5 days, within 6 days, within 7 days, within 2 weeks, within 3 weeks, within 4 weeks, within 1 month, within 2 months, within 3 months, within 4 months, within 5 months, within 6 months, within 7 months, within 8 months, within 9 months, within 10 months, within 11 months, within 12 months, within 13 months, within 14 months, within 15 months, within 16 months, within 17 months, within 18 months, within 19 months, within 20 months, within 21 months, within 22 months, within 23 months, or within 24 months after rotator cuff repair. Since 3 to 6 months after rotator cuff repair is the most important period for healing, the timing of attaching or fixing the nanofiber sheet is an important factor in healing after rotator cuff repair. The timing of attaching or fixing the nanofiber sheet may vary depending on the age, sex, and weight of the subject to be treated within the above-described range, the specific disease or pathological condition to be treated, the severity of the disease or pathological condition, the route of administration, and the judgment of the prescriber.

The sheet, according to an aspect of the present invention, may be locally attached or fixed to a rotator cuff tear site.

The sheet, according to an aspect of the present invention, may enhance at least one selected from the group consisting of vascular permeability, cell density, continuity, density, orientation of collagen fibers of rotator cuff, and maturation in tendon to bone junctions. According to an embodiment of the present invention, attachment of the nanofiber sheet containing recombinant parathyroid hormone, compared to direct systemic or local administration of recombinant parathyroid hormone, enhances vascular permeability, cell density, continuity, density, orientation of collagen fibers of rotator cuff, and maturation in tendon to bone junctions, thereby obtaining an excellent effect of healing of rotator cuff tears (Experimental Example 1-2 and Experimental Example 2-3).

The sheet, according to an aspect of the present invention, may increase the expression level of one or more genes selected from the group consisting of collagen type I alpha 1 (COL1A1), collagen type Ill alpha 1 (COL3A1), bone morphogenetic protein 2 (BMP-2), scleraxis (SCX), SRY-box 9 (SOX9), and aggrecan (ACAN), compared to the sheet unattached group or before sheet attachment. Specifically, in the sheet according to an aspect of the present invention, the expression level of one or more genes selected from the group consisting of COL1A1, COL3A1, BMP-2, SCX, SOX9, and ACAN is increased by 1% or more, 2% or more, 3% or more, 4% or more, 5% or more, 6% or more, 8% or more, 10% or more, 12% or more, 14% or more, 16% or more, 18% or more, 20% or more, 22% or more, 24% or more, 26% or more, 28% or more, 30% or more, or 32% or more, compared to the sheet unattached group or before sheet attachment. According to an embodiment of the present invention, attachment of the nanofiber sheet containing recombinant parathyroid hormone, compared to direct systemic or local administration of recombinant parathyroid hormone, enhances vascular permeability, cell density, continuity, density, orientation of collagen fibers of rotator cuff, and maturation in tendon to bone junctions, thereby obtaining an excellent effect of healing of rotator cuff tears (Experimental Example 1-2 and Experimental Example 2-3).

The sheet, according to an aspect of the present invention, may be an electrospun nanofiber sheet.

In another aspect, the present invention provides a method of manufacturing a nanofiber sheet for healing of rotator cuff tears, the method including incorporating a composition including teriparatide, isomers thereof, pharmaceutically acceptable salts thereof, hydrates thereof, or solvates thereof as an active ingredient in a nanofiber sheet. The description of the nanofiber sheet for healing of rotator cuff tears may be applied to the manufacturing method.

In the manufacturing method according to an aspect of the present invention, the present invention provides a method of manufacturing a nanofiber sheet for healing of rotator cuff tears, the method comprising forming a nanofiber sheet by electrospinning an electrospinning solution; and impregnating the formed sheet with a composition containing teriparatide, isomers thereof, pharmaceutically acceptable salts thereof, hydrates thereof, or solvates thereof as an active ingredient.

The electrospinning solution according to an aspect of the present invention may be prepared by adding to the organic solution at least one selected from the group consisting of polycaprolactone (PCL), collagen, gelatin, elastin, chitosan, silk fibroin, alginate, poly(glycolic acid) (PGA), poly(lactic-co-glycolic acid) (PLGA), poly(L-lactic acid)(PLLA), poly(L-lactic acid)/collagen (PLLA/CL), polyvinyl alcohol (PVA), and polyethylene oxide (PEO).

The polycaprolactone (PCL) according to an aspect of the present invention as a polyester-based biodegradable polymer polymerized from caprolactone (CL) is known as a polymer with excellent biodegradability and biocompatibility, and thus can be used for drug delivery and research in various medical fields.

The organic solvent according to an aspect of the present invention may be any one selected from the group consisting of 2,2,2-trifluoroethanol, chloroform, dichloromethane, tetrahydrofuran, dimethylformamide, and dimethylsulfoxide, but is not limited thereto. In the manufacturing method according to an aspect of the present invention, the present invention provides a method of manufacturing a nanofiber sheet for healing of rotator cuff tears, the method including forming the nanofibers containing the composition by using the external nozzle discharging electrospinning solution, and using the internal nozzle discharging a composition containing teriparatide, isomers thereof, pharmaceutically acceptable salts thereof, hydrates thereof, or solvates thereof as an active ingredient. For example, with the above manufacturing method, it is possible to produce nanofibers having a core-shell structure in which a nanofiber is in the form of a shell, and the composition is in the form of a core.

The electrospinning solution is as described above.

Hereinafter, the configuration and effects of the present invention will be described in more detail by way of examples and experimental examples. However, the following examples and experimental examples are provided only for illustrative purposes to aid understanding of the present invention, and the scope of the present invention is not limited thereto.

In the following examples and experimental examples, animal care and all experimental procedures were performed in accordance with guidelines approved by the Animal Care and Use Committee of the Clinical Research Institute of the senior author (J.H.O.) (IACUC No. BA-2006-297-048-01). In addition, for multi-group analysis, data from PCR and biomechanical testing were evaluated using the Kruskal-Wallis test, followed by a post-hoc Mann-Whitney U test using Bonferroni correction. The histological categorical variables were analyzed using a chi-square test for trend. All statistical analyzes were performed by an expert statistician in a blinded manner using SPSS v.23.0 (IBM, Armonk, NY, USA), and the P value <0.05 was considered as a statistically significant adversary.

### [Preparation Example 1] Manufacture of Nanofiber Sheet 1 (rhPTH-soaked Nanofiber Sheet) for Healing of Rotator Cuff Tears

### 1. Fabrication of 3D Printed Nanofiber Sheet

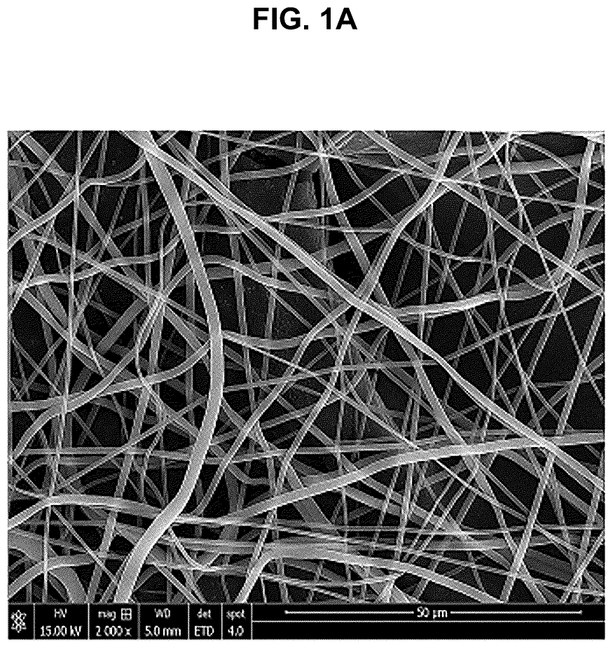

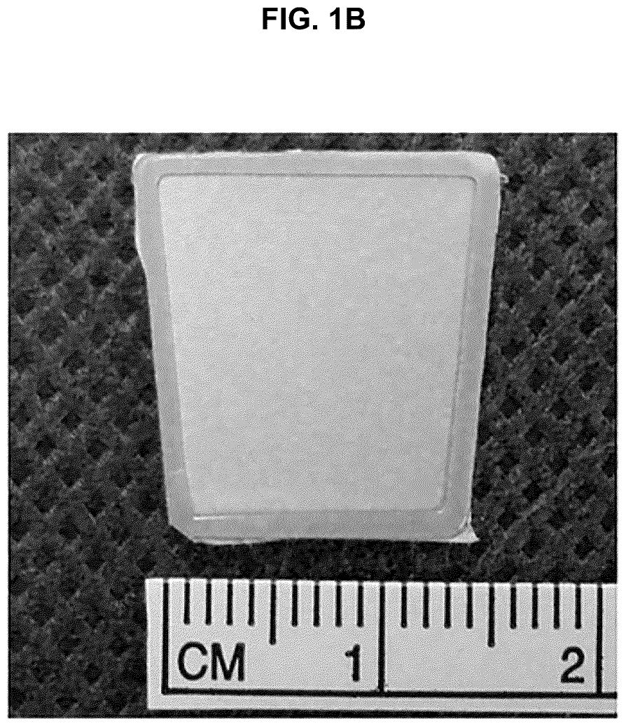

Polycaprolactone (PCL; MW, 80000; Sigma-Aldrich, St. Louis, MO, USA) solution dissolved in a mixture of dichloromethane (Samchun Pure Chemical, Republic of Korea) and dimethylformamide (Junsei Chemical, Japan) in a volume ratio of 75:25 as an electrospinning material was used. The PCL solution of 14% (w/v) was injected through a 23-G (inner diameter of 0.34 mm) metal needle at a rate of 60 uL/min, and a voltage of 20 kV per 200 mm tip-to-collector distance was applied [Teo W E, Ramakrishna S. A review on electrospinning design and nanofiber assemblies. Nanotechnology 2006; 17(14):R89-R106. doi:10.1088/0957-4484/17/14/R01]. At high electric fields, the solution was splashed from the needle, forming numerous nano-sized fibers. The spun fibers were discharged from the needle and landed on the hard surface of the counter-electrode collector. The time for electrospinning was set to 100 seconds. The shape of electrospun nanofibers (FIG. 1A) was confirmed using a scanning electron microscope The PCL filament feedstock was then printed using a homemade 3D printing system based on a material extrusion mechanism. It was treated with a microstructured framework fused with the nanofiber sheet (FIG. 11B). The framework stabilized the structure of the nanofiber sheet.

### 2. Selection of Carrier

Hyaluronic acid (HA) was chosen as a carrier for loading rhPTH. It is a high-viscosity polysaccharide that plays an important role in organizing ECM in the human body, and may be a desirable biopharmaceutical agent due to its anti-inflammatory effects and stimulation of tendon-to-bone healing [Li H, Ge Y, Zhang P, Wu L, Chen S. The effect of layer-by-layer chitosan-hyaluronic acid coating on graft-to-bone healing of a poly(ethylene terephthalate) artificial ligament. J Biomater Sci Polym Ed 2012; 23(1-4):425-38. doi:10.1163/092050610X551989]. The HA has also been researched for use as a carrier for drugs, cells, and more recently, proteins [Huang G, Huang H. Application of hyaluronic acid as carriers in drug delivery. Drug Deliv 2018; 25(1):766-72. doi:10.1080/10717544.2018.1450910; Mero A, Campisi M, Caputo M, Cuppari C, Rosato A, Schiavon O, et al. Hyaluronic Acid as a Protein Polymeric Carrier: An Overview and a Report on Human Growth Hormone. Curr Drug Targets 2015; 16(13):1503-11. doi:10.2174/1389450116666150107151906]. The protein should be released for a long period of time without being denatured [Bayer I S. Hyaluronic Acid and Controlled Release: A Review. Molecules 2020; 25(11). doi:10.3390/molecules25112649]. However, it has been reported that nanofiber scaffolds show rapid burst release in the early stages of implantation. Therefore, the HA plays a very important role as a rhPTH carrier to maintain rhPTH concentration for a long period of time in the 3D printed nanofiber sheet [Gentile P, Nandagiri V K, Pabari R, Daly J, Tonda-Turo C, Ciardelli G, et al. Influence of Parathyroid Hormone-Loaded PLGA Nanoparticles in Porous Scaffolds for Bone Regeneration. Int J Mol Sci 2015; 16(9):20492-510. doi:10.3390/ijms160920492].

3. Fabrication of 3D Printed rhPTH-Soaked Nanofiber Sheet

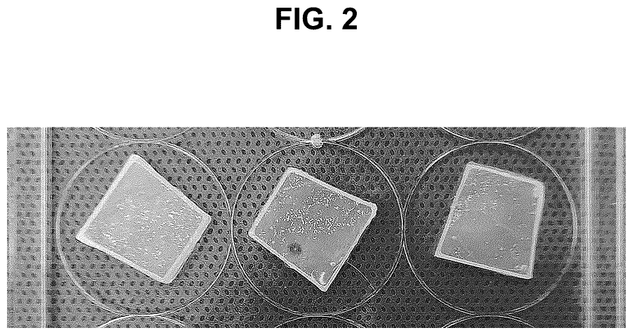

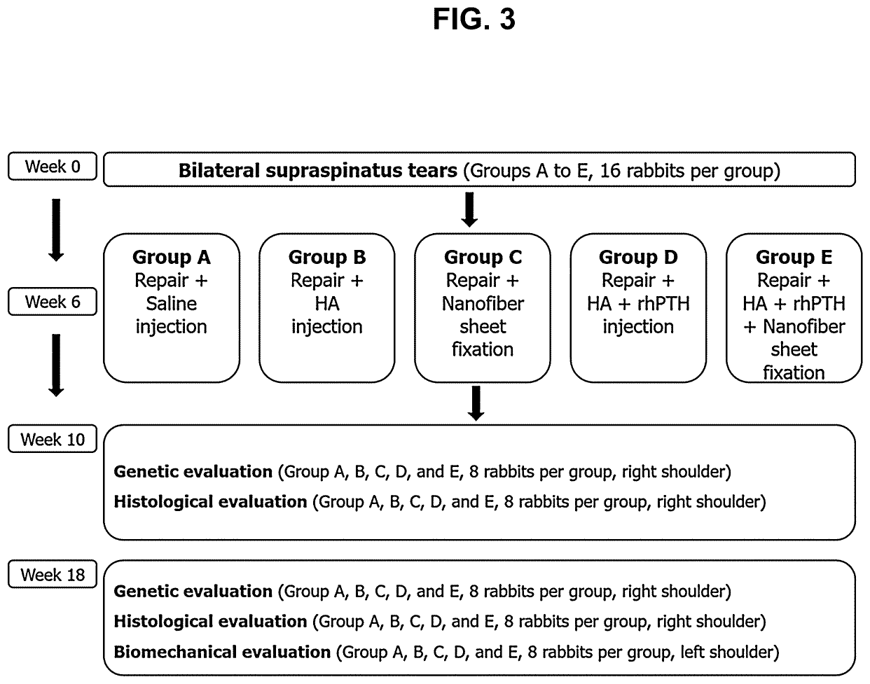

After 3D printing, the printed nanofiber sheet was sterilized with 70% ethanol for at least 30 minutes [Horakova J, Klicova M, Erben J, Klapstova A, Novotny V, Behalek L, et al. Impact of Various Sterilization and Disinfection Techniques on Electrospun Poly-epsilon-caprolactone. ACS Omega 2020; 5(15):8885-92. doi:10.1021/acsomega.0c00503]. Thereafter, the 3D printed nanofiber sheet was thinly wetted with a mixture of 20 μg rhPTH (Forteo; Eli Lilly, Indianapolis, IN, USA) and 0.1 mL HA (Synovian; LG Life Sciences, Seoul, Republic of Korea)(FIG. 2).

### [Example 1] Topical Attachment of Nanofiber Sheet for Healing of Rotator Cuff Tears

In order to confirm the rotator cuff tear healing effect of the nanofiber sheet for healing of rotator cuff tears prepared in Preparation Example 1, animal experiments were performed as follows.

### [Example 1-1] Allocation of Rabbits

The sample size was determined by power analysis as previously described [Kwon J, Kim Y H, Rhee S M, Kim T I, Lee J, Jeon S, et al. Effects of Allogenic Dermal Fibroblasts on Rotator Cuff Healing in a Rabbit Model of Chronic Tear. Am J Sports Med 2018; 46(8):1901-8. doi:10.1177/0363546518770428; Uhthoff H K, Seki M, Backman D S, Trudel G, Himori K, Sano H. Tensile strength of the supraspinatus after reimplantation into a bony trough: an experimental study in rabbits. J Shoulder Elbow Surg 2002; 11(5):504-9. doi:10.1067/mse.2002.126760]. At the minimum sample size, 8 rabbits were required to detect significant differences in final defect load (mean difference: 90 N; standard deviation: 40 N; α-error: 0.05; β-error: 0.2; dropout rate: 25%). 80 female New Zealand white rabbits (mean age: 6 months; weight: 3.5 to 4.0 kg) were randomly allocated to 5 groups (16 rabbits per group): group A (suture and saline injection), group B (suture and HA injection), group C (suture and 3D printed nanofiber sheet fixation), group D (suture and rhPTH and HA injection), and group E (suture and 3D printed rhPTH- and HA-soaked nanofiber sheet fixation). All rabbits underwent bilateral surgery, including a total of 160 shoulders thereof. Each sample from half of the rabbits (8 rabbits per group) underwent gene expression analysis and histological evaluation at 4 weeks after suturing. Each sample from the right shoulder of the remaining rabbits (8 rabbits per group) underwent gene expression analysis and histological evaluation at 12 weeks after suturing, and samples from the left shoulder thereof underwent biomechanical evaluation at 12 weeks after suturing.

### [Example 1-2] Surgical Procedure

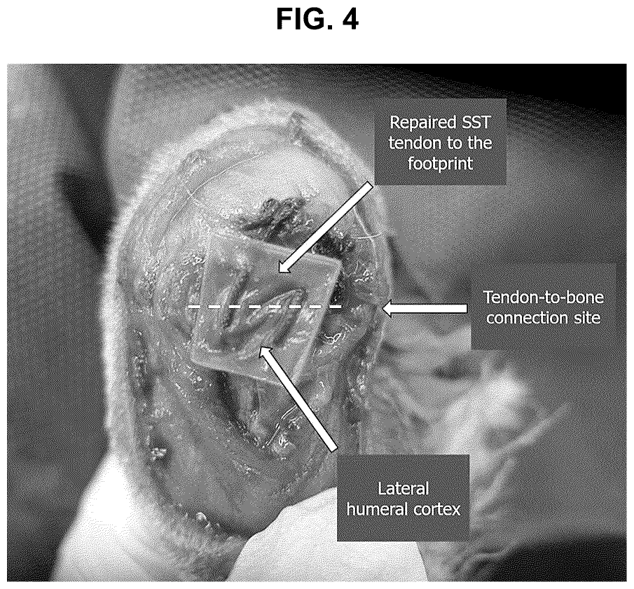

Under anesthesia, a longitudinal incision was made on the side of both shoulders, and the deltoid muscle was contracted to expose the supraspinatus tendon of the rabbit of Example 1. According to the previously reported chronic RCT model generation process [Chung S W, Park H, Kwon J, Choe G Y, Kim S H, Oh J H. Effect of Hypercholesterolemia on Fatty Infiltration and Quality of Tendon-to-Bone Healing in a Rabbit Model of a Chronic Rotator Cuff Tear: Electrophysiological, Biomechanical, and Histological Analyses. Am J Sports Med 2016; 44(5):1153-64. Doi:10.1177/0363546515627816], to prevent adhesion to the surrounding soft tissue, the supraspinatus tendon was cut using a sharp scalpel at the footprint of the greater tuberosity and wrapped with a 10 mm long silicone Penrose drain (outer diameter: 8 mm; Yushin Corp, Republic of Korea). Six weeks after the induction of supraspinatus tear, the Penrose drain around the torn supraspinatus tendon was removed, and the isolated supraspinatus tendon was sutured using a 2-0 Ticron (Tyco, Waltham, MA, USA) at the footprint of the greater tuberosity of the humerus using the transosseous repair technique. Specifically, the soft tissue surrounding the exposed greater tuberosity was excised using a scalpel blade to create a bone hemorrhage. Two interosseous tunnels were created at the articular margin of the supraspinatus footprint to the lateral brachial cortex. For reattachment of the torn tendon, the suture end was passed through the bone tunnel and tied to reconnect the supraspinatus tendon to the footprint. Thereafter, 0.1 mL saline, 0.1 mL HA, a mixture of 0.1 mL HA and 20 ug rhPTH were injected into the proximal joints of the supraspinatus on both sides in group A, group B, and group D, respectively. In group C, the 3D printed nanofiber sheet was placed in the tendon to bone sutures of both shoulders. In group E, the 3D printed rhPTH-soaked nanofiber sheet was placed in the same way as group C (FIG. 4). The wound was covered in several layers, and the several layers were absorbed into the tissue. After surgery, the rabbits were individually housed and were not weight-bearing restricted or immobilized in any way. Cefazolin was intramuscularly injected every 24 hours for 3 days at 30 mg/kg after surgery to prevent infection after surgery.

However, data from 8 rabbits were excluded from the final analysis. One rabbit from group A, two rabbits from group B, and two rabbits from group D showed active infection with purulent discharge after 4 weeks of suturing. At the same time, one rabbit from group C and two rabbits from group E died due to anesthesia accidents. At the final evaluation, no group of rabbits showed supraspinatus tendon dehiscence at the footprint.

### [Experimental Example 1-1] Gene Expression Level Comparison

Experiment After Attaching Nanofiber Sheet for Healing of Rotator Cuff Tears After placing (attaching) the nanofiber sheet for healing of rotator cuff tears prepared in Preparation Example 1 to a rabbit with a rotator cuff tear according to Example 1, in order to confirm the tear healing effect of the sheet, quantitative real-time polymerase chain reaction (qRT-PCR) was performed as follows.

Specifically, 40 rabbits for each time point (4 weeks and 12 weeks after suturing) were anesthetized and humanely sacrificed by intravenous injection of saturated potassium chloride solution (2 mmol/kg). The minimal sample (3×3 mm²) of sutured supraspinatus tendon was taken from each right shoulder for mRNA expression analysis, immediately frozen in liquid nitrogen, and stored at −80° C. [Lee J H, Kim Y H, Rhee S M, Han J, Jeong H J, Park J H, et al. Rotator Cuff Tendon Healing Using Human Dermal Fibroblasts: Histological and Biomechanical Analyses in a Rabbit Model of Chronic Rotator Cuff Tears. Am J Sports Med 2021; 49(13):3669-79. doi:10.1177/03635465211041102]. RNA was extracted using RNeasy Mini Kit columns (Qiagen, Hilden, Germany) using the manufacturer's protocol. The total RNA from tendon samples was homogenized in tubes containing beads using TRIzol reagent (Invitrogen, Carlsbad, CA, USA). 1 μg of total RNA was reverse transcribed into complementary DNA using Maxime R T PreMix (iNtRON, Bio Inc., Sungnam, Republic of Korea). qRT-PCR was performed using the QuantStudio 6 Flex Real-Time PCR System (Applied Biosystems, Foster City, CA, USA) with SYBR Green PCR master mix (Applied Biosystems). The following protocol was used: repeating denaturation at 95° C. for 10 minutes, followed by 40 cycles of denaturation at 95° C. for 15 seconds and annealing at 58° C. for 60 seconds without extension to calculate values of the linear phase. The relative gene expression levels were analyzed using the 2-ΔΔCT method using glyceraldehyde-3-phosphate dehydrogenase (GAPDH) as an internal reference [Livak K J, Schmittgen T D. Analysis of relative gene expression data using real-time quantitative PCR and the 2(-Delta Delta C(T)) Method. Methods 2001; 25(4):402-8.]. COL1A1 and COL3A1 primarily encode the major components of type I collagen and type Ill collagen, respectively. BMP-2 plays an important role in bone and tendon development, and SCX is mainly expressed in precursor cells and cells of all tendon tissues. SOX9 and ACAN are essential transcription factors required for chondrocyte differentiation and cartilage formation, and the primer sequences used in qRT-PCR are shown in Table 1 below.

TABLE 1

| mRNA | | |
|---|---|---|
| Gene | Forward | Reverse |
| COL1A1 | 5′-GGTCTTCTGCGA | 5′-CCACACGTGCTT |
| | CATGGACA-3′ | CTTCTCCT-3′ |
| | (SEQ ID No: 1) | (SEQ ID No: 2) |
| COL3A1 | 5′-GCTCTGCTTCAT | 5′-ATATTTGGCACG |
| | CCCACTGT-3′ | GTTCGGGT-3′ |
| | (SEQ ID No: 3) | (SEQ ID No: 4) |
| BMP-2 | 5′-GGGTGGAACGAC | 5′-TGCACGATGGCA |
| | TGGATTGT-3′ | TGGTTAGT-3′ |
| | (SEQ ID No: 5) | (SEQ ID No: 6) |
| SCX | 5′-ACAGATCTGCAC | 5′-CCGTGACTCTTC |
| | CTTCTGCC-3′ | AGTGGCAT-3′ |
| | (SEQ ID No: 7) | (SEQ ID No: 8) |
| SOX9 | 5′-GCCCAGAAGAGC | 5′-GGTACCAGTTGC |
| | CTCAAAGT-3′ | CTTCAGCT-3′ |
| | (SEQ ID No: 9) | (SEQ ID No: 10) |
| ACAN | 5′-GGATCTACCGCT | 5′-GTGGAGATGGCC |
| | GTGAGGTG-3′ | CGATAGTG-3′ |
| | (SEQ ID No: 11) | (SEQ ID No: 12) |
| GAPDH | 5′-GGAATCCACTGG | 5′-GGTTCACGCCCA |
| | CGTCTTCA-3′ | TCACAAAC-3′ |
| | (SEQ ID No: 13) | (SEQ ID No: 14) |

COL1A1 (collagen type I alpha 1);
COL3A1 (collagen type III alpha 1);
BMP-2 (bone morphogenetic protein 2);
SCX (scleraxis);
SOX9 (SRY-box 9);
ACAN (aggrecan);
GAPDH (glyceraldehyde-3-phosphatate dehydrogenase)

The entire procedure was performed by an independent analyst ignorant of group allocations, and the results are shown in Table 2 below.

TABLE 2

| | After 4 weeks of suture | | | | | | After 12 weeks of suture | | | | | |
|---|---|---|---|---|---|---|---|---|---|---|---|---|
| | Group | Group | Group | Group | Group | | Group | Group | Group | Group | Group | |
| | A | B | C | D | E | P | A | B | C | D | E | P |
| | (n = 7) | (n = 6) | (n = 7) | (n = 6) | (n = 6) | value | (n = 8) | (n = 8) | (n = 8) | (n = 8) | (n = 8) | value |
| COL1A1 | 0.89 ± | 0.92 ± | 0.90 ± | 0.92 ± | 1.18 ± | 0.008* | 0.79 ± | 0.83 ± | 0.75 ± | 0.92 ± | 0.94 ± | .074 |
| | 0.06 | 0.08 | 0.06 | 0.09 | 0.08 | | 0.12 | 0.09 | 0.22 | 0.06 | 0.06 | |
| COL3A1 | 7.33 ± | 8.26 ± | 8.19 ± | 8.39 ± | 8.69 ± | .103 | 2.29 ± | 2.02 ± | 2.69 ± | 2.63 ± | 3.21 ± | .138 |
| | 0.59 | 0.94 | 0.75 | 1.15 | 0.47 | | 0.96 | 0.62 | 0.97 | 1.08 | 1.50 | |
| BMP-2 | 0.91 ± | 0.97 ± | 1.04 ± | 1.03 ± | 1.05 ± | .334 | 0.72 ± | 0.62 ± | 0.83.± | 0.67 ± | 0.59 ± | .283 |
| | 0.09 | 0.10 | 0.19 | 0.09 | 0.09 | | 0.19 | 0.09 | 0.38 | 0.10 | 0.13 | |
| SCX | 1.32 ± | 1.33 ± | 1.38 ± | 1.38 ± | 1.41 ± | .453 | 1.29 ± | 1.28 ± | 1.34 ± | 1.28 ± | 1.24 ± | .963 |
| | 0.10 | 0.05 | 0.29 | 0.07 | 0.08 | | 0.38 | 0.46 | 0.33 | 0.51 | 0.45 | |
| SOX9 | 1.14 ± | 1.11 ± | 1.12 ± | 1.15 ± | 1.22 ± | .665 | 0.69 ± | 0.65 ± | 0.65 ± | 0.64 ± | 0.68 ± | .975 |
| | 0.12 | 0.10 | 0.22 | 0.07 | 0.12 | | 0.19 | 0.08 | 0.29 | 0.12 | 0.24 | |
| ACAN | 1.14 ± | 1.13 ± | 1.21 ± | 1.14 ± | 1.23 ± | .955 | 2.24 ± | 1.91 ± | 2.41 ± | 2.56 ± | 2.38 ± | .349 |
| | 0.20 | 0.25 | 0.22 | 0.30 | 0.25 | | 0.62 | 0.64 | 0.52 | 0.71 | 0.48 | |

Saline injection in Group A; HA injection in Group B; 3D printed nanofiber sheet fixation in Group C; HA and rhPTH injection in Group D; 3D printed, rhPTH-soaked nanofiber sheet fixation in Group E. Data are expressed as mean ± SD.
COL1A1 (collagen type 1 alpha 1); COL3A1 (collagen type III alpha 1); BMP-2 (bone morphogenetic protein 2); SCX (scleraxis); SOX9 (SRY-box 9); ACAN (aggrecan).
The relative target gene expression ratio compared to glyceraldehyde-3-phosphate dehydrogenase (GAPDH) was quantified.
*Significantly different: there was a significant difference in COL1A1 expression levels between groups A and E (P = 001), between groups B and E (P = .004), and between groups C and E (P = .004).

As shown in Table 2, group E showed the highest COL1A1 mRNA expression level among all groups (P=0.008). However, the expression levels did not differ between the groups after 12 weeks of suturing (P=0.074). There were no significant differences in the mRNA expression levels of COL3A1, BMP-2, SCX, SOX9, and ACAN at 4 or 12 weeks after suturing (all P>0.05).

It was found that the nanofiber sheet for healing of rotator cuff tears according to an aspect of the present invention has an excellent effect of healing torn rotator cuff by increasing the mRNA expression level of COL1A1 encoding type I collagen.

### [Experimental Example 1-2] Histological Evaluation

After placing (attaching) the nanofiber sheet for healing of rotator cuff tears prepared in Preparation Example 1 to a rabbit with a rotator cuff tear according to the above example, the histological evaluation by the following method to confirm the tear healing effect of the sheet was performed.

Specifically, at 4 and 12 weeks after suturing, supraspinatus tendon samples were processed for the histological evaluation as previously published [Chung S W, Park H, Kwon J, Choe G Y, Kim S H, Oh J H. Effect of Hypercholesterolemia on Fatty Infiltration and Quality of Tendon-to-Bone Healing in a Rabbit Model of a Chronic Rotator Cuff Tear: Electrophysiological, Biomechanical, and Histological Analyses. Am J Sports Med 2016; 44(5):1153-64. doi:10.1177/0363546515627816; Kwon J, Kim Y H, Rhee S M, Kim T I, Lee J, Jeon S, et al. Effects of Allogenic Dermal Fibroblasts on Rotator Cuff Healing in a Rabbit Model of Chronic Tear. Am J Sports Med 2018; 46(8):1901-8. doi:10.1177/0363546518770428; Oh J H, Chung S W, Kim S H, Chung J Y, Kim J Y. 2013 Neer Award: Effect of the adipose-derived stem cell for the improvement of fatty degeneration and rotator cuff healing in rabbit model. J Shoulder Elbow Surg 2014; 23(4):445-55. doi:10.1016/j.jse.2013.07.054]. The tissues were fixed overnight in 10% neutral-buffered formalin for hematoxylin and eosin staining, embedded in paraffin, and cut into 5 mm thick blocks to assess the tendon to bone healing at the suture site. Masson's trichrome staining was used to determine the continuity, orientation and density of collagen fibers, which was performed on paraffin sections. Each slide (10 scan sections including slides) was analyzed by one orthopedic surgeon and one musculoskeletal pathologist in a randomized and blinded manner to minimize bias. For quantitative analysis, the mean value of 10 measured values was used.

The histological parameters including the continuity, orientation and density of collagen fibers and tendon to bone interface maturity were each graded semi-quantitatively using a 4-level system (0 to 3 grades) as previously reported [Chung S W, Park H, Kwon J, Choe G Y, Kim S H, Oh J H. Effect of Hypercholesterolemia on Fatty Infiltration and Quality of Tendon-to-Bone Healing in a Rabbit Model of a Chronic Rotator Cuff Tear: Electrophysiological, Biomechanical, and Histological Analyses. Am J Sports Med 2016; 44(5):1153-64. doi:10.1177/0363546515627816]. The continuity and orientation of the collagen fibers were indicated in percentages as follows: grade 0: 0%-24%; grade 1: 25%-49%; grade 2: 50%-74%; and grade 3: 75%-100%. The density of collagen fibers was classified as very loose, loose, dense, and very dense (0 to 3 grades, respectively). The tendon to bone interface maturity was graded from 0 to 3, which corresponded to poor, mild, moderate or dense, respectively. For statistical analysis, response levels were digitized as 1 for grade 0, 2 for grade 1, 3 for grade 2, and 4 for grade 3.

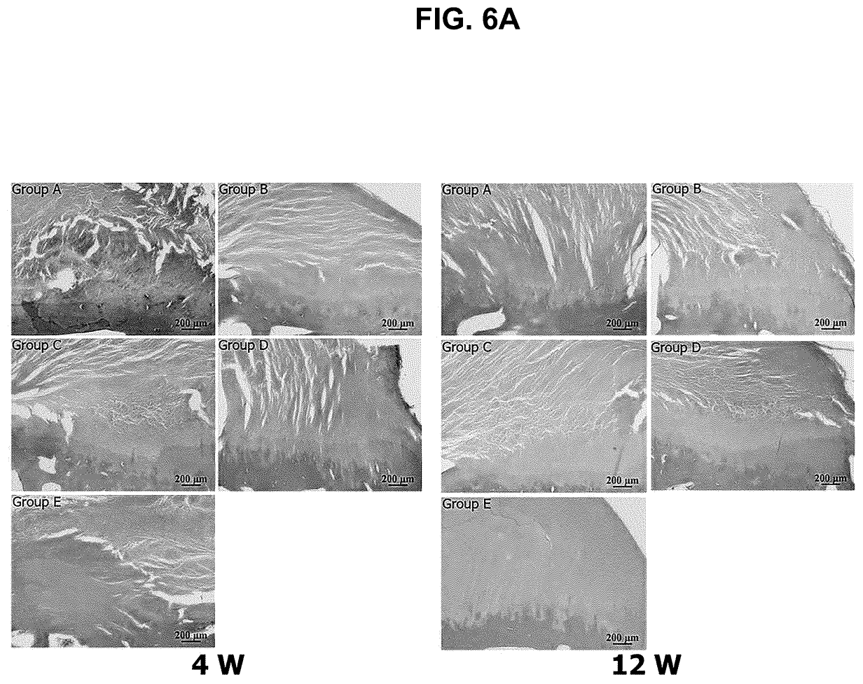

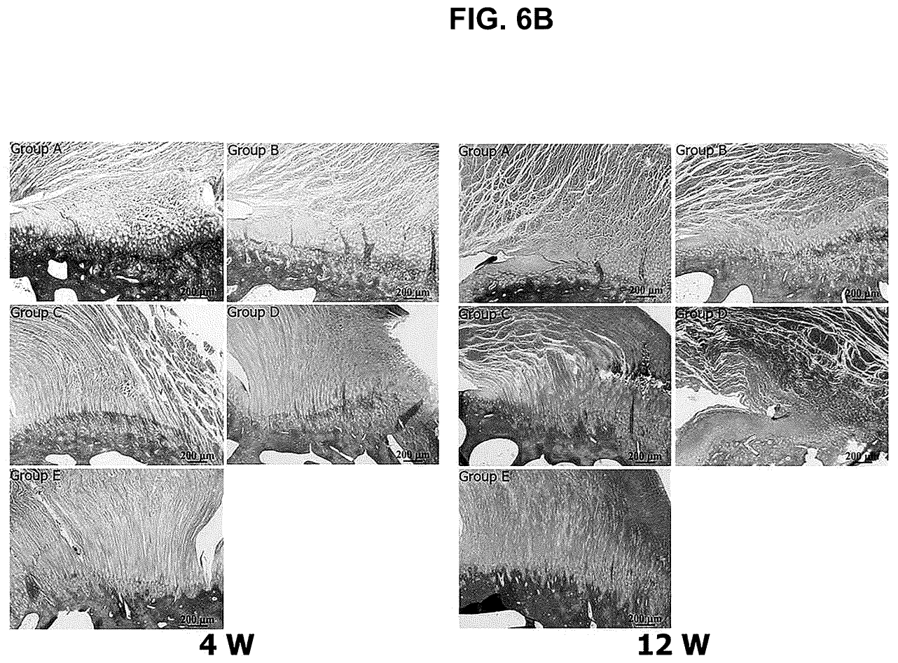

The images were captured and obtained by using an Eclipse Ci-L microscope (Nikon, Tokyo, Japan) and using a Nikon DS-U3 and NIS Elements BR 5.2 acquisition software (Nikon), and results of histological analysis according to the semi-quantitative grading system are shown in Table 3 and FIG. 6.

TABLE 3

| | After 4 weeks of suture | | | | | | | | | | | | | | | | | | | | | After 12 weeks of suture | |
|---|---|---|---|---|---|---|---|---|---|---|---|---|---|---|---|---|---|---|---|---|---|---|---|
| | Group A | | | | Group B | | | | Group C | | | | Group D | | | | Group E | | | | | Group A | |
| | (n = 7) | | | | (n = 6) | | | | (n = 7) | | | | (n = 6) | | | | (n = 6) | | | | P | (n = 8) | |
| Parameter | G0 | G1 | G2 | G3 | G0 | G1 | G2 | G3 | G0 | G1 | G2 | G3 | G0 | G1 | G2 | G3 | G0 | G1 | G2 | G3 | value | G0 | G1 |
| Continuity | 6 | 1 | 0 | 0 | 5 | 1 | 0 | 0 | 4 | 3 | 0 | 0 | 2 | 4 | 0 | 0 | 2 | 4 | 0 | 0 | 0.145 | 5 | 5 |
| of collagen | | | | | | | | | | | | | | | | | | | | | | | |
| fibers | | | | | | | | | | | | | | | | | | | | | | | |
| Orientation | 5 | 2 | 0 | 0 | 4 | 2 | 0 | 0 | 5 | 2 | 0 | 0 | 3 | 3 | 0 | 0 | 2 | 4 | 0 | 0 | 0.575 | 3 | 1 |
| of collagen | | | | | | | | | | | | | | | | | | | | | | | |
| fibers | | | | | | | | | | | | | | | | | | | | | | | |
| Density of | 5 | 2 | 0 | 0 | 3 | 3 | 0 | 0 | 4 | 3 | 0 | 0 | 2 | 4 | 0 | 0 | 1 | 5 | 0 | 0 | 0.325 | 5 | 5 |
| collagen | | | | | | | | | | | | | | | | | | | | | | | |
| fibers | | | | | | | | | | | | | | | | | | | | | | | |
| TTB junction | 6 | 1 | 0 | 0 | 5 | 1 | 0 | 0 | 4 | 3 | 0 | 0 | 3 | 3 | 0 | 0 | 2 | 4 | 0 | 0 | 0.251 | 5 | 3 |
| maturation | | | | | | | | | | | | | | | | | | | | | | | |

| | After 12 weeks of suture | | | | | | | | | | | | | | | | | | |
|---|---|---|---|---|---|---|---|---|---|---|---|---|---|---|---|---|---|---|---|
| | Group A | | Group B | | | | Group C | | | | Group D | | | | Group E | | | | |
| | (n = 8) | | (n = 8) | | | | (n = 8) | | | | (n = 8) | | | | (n = 8) | | | | P |
| Parameter | G2 | G3 | G0 | G1 | G2 | G3 | G0 | G1 | G2 | G3 | G0 | G1 | G2 | G3 | G0 | G1 | G2 | G3 | value |
| Continuity | 0 | 0 | 4 | 3 | 1 | 0 | 2 | 4 | 2 | 0 | 2 | 2 | 4 | 0 | 0 | 0 | 3 | 5 | 0.001* |
| of collagen | | | | | | | | | | | | | | | | | | | |
| fibers | | | | | | | | | | | | | | | | | | | |
| Orientation | 4 | 0 | 1 | 3 | 3 | 1 | 2 | 3 | 2 | 1 | 1 | 2 | 4 | 1 | 0 | 1 | 4 | 3 | 0.559 |
| of collagen | | | | | | | | | | | | | | | | | | | |
| fibers | | | | | | | | | | | | | | | | | | | |
| Density of | 0 | 0 | 3 | 4 | 1 | 3 | 3 | 3 | 2 | 0 | 2 | 3 | 3 | 0 | 0 | 0 | 3 | 5 | 0.001* |
| collagen | | | | | | | | | | | | | | | | | | | |
| fibers | | | | | | | | | | | | | | | | | | | |
| TTB junction | 0 | 0 | 4 | 3 | 0 | 0 | 2 | 4 | 2 | 0 | 2 | 2 | 4 | 0 | 0 | 0 | 4 | 4 | 0.003* |
| maturation | | | | | | | | | | | | | | | | | | | |

Grades: grade 0, none or minimal (0%-24%); grade 1, mild (25%-49%); grade 2, moderate (50%-74%); and grade 3, marked (75%-100%). Saline injection in Group A; HA injection in Group B; 3D printed nanofiber sheet fixation in Group C; HA and rhPTH injection In Group D; 3D printed, rhPTH-soaked nanofiber sheet fixation. in Group E.
*Significantly different.

As shown in Table 3 and FIG. 6, there was no apparent difference in any parameter between the groups after 4 weeks of suturing (P>0.05). However, the continuity of the collagen fibers was greater in group E than in the other groups after 12 weeks of suturing (P=0.001) (FIG. 6A). In addition, group E showed denser collagen fibers and more mature tendon to bone junctions than the other groups (FIG. 6B). The group E had the higher orientation of collagen fibers, but there was no significant difference compared to other groups (P=0.559).

Therefore, the nanofiber sheet for healing of rotator cuff tears according to an aspect of the present invention, compared to direct administration of recombinant parathyroid hormone, enhances at least one selected from the group consisting of the continuity, the orientation, and the density of collagen fibers, and the maturation in tendon-to-bone junctions, and thus has an excellent effect of healing a torn rotator cuff or a tear of a rotator cuff suture site or reducing re-tear after rotator cuff repair.

### [Experimental Example 1-3] Biomechanical Evaluation

After placing (attaching) the nanofiber sheet for healing of rotator cuff tears prepared in Preparation Example 1 to a rabbit with a rotator cuff tear according to Example 1, in order to confirm the tear healing effect of the sheet, the biomechanical evaluation was performed in the following manner.

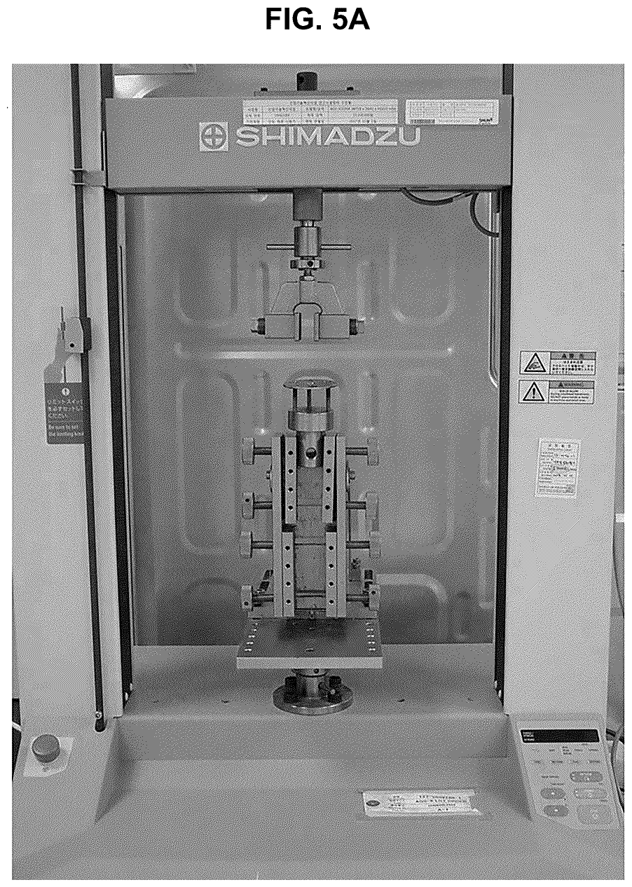

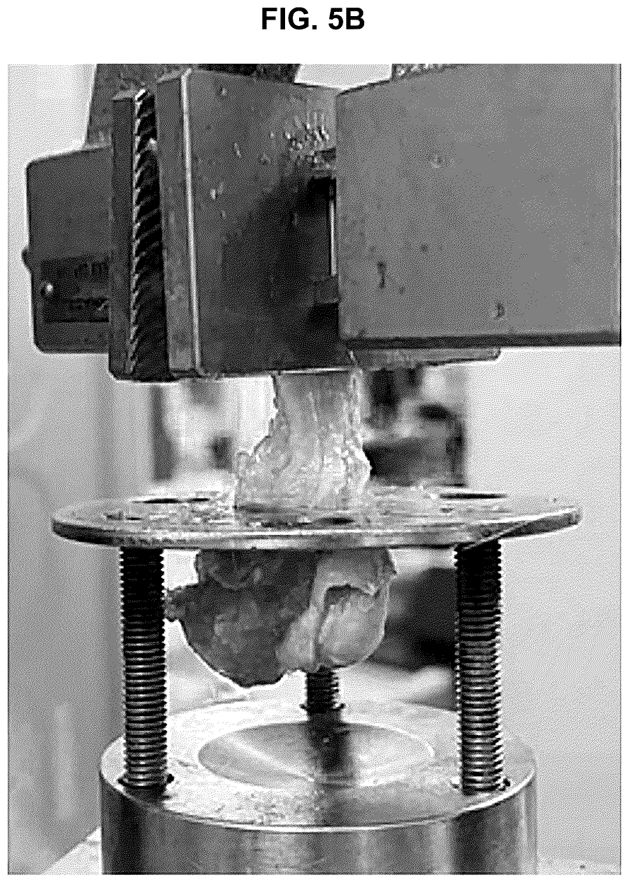

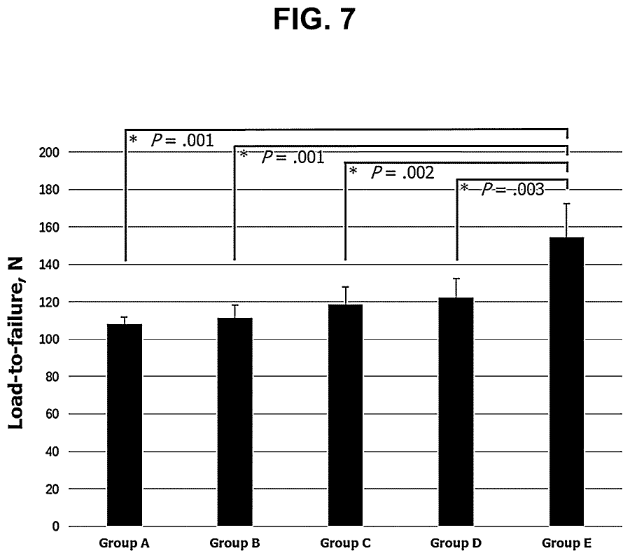

Specifically, at 12 weeks after suturing, under appropriate anesthesia and euthanasia, 40 samples (8 samples per group) with attached humeral head and supraspinatus tendon unit were collected for the biomechanical evaluation using a universal material testing machine (AGS-X; Shimadzu, Kyoto, Japan). The machine consists of two sub-sections including the humeral head and tendon fixation unit. To form the right angle, the supraspinatus tendon was clamped to this system along the anatomical direction to allow tensile loading (FIG. 5). Parameters of the biomechanical evaluation were mode of failure (insertional tear: mid-substance tear) and load-to-failure, which were measured as previously described [Oh J H, Chung S W, Kim S H, Chung J Y, Kim J Y. 2013 Neer Award: Effect of the adipose-derived stem cell for the improvement of fatty degeneration and rotator cuff healing in rabbit model. J Shoulder Elbow Surg 2014; 23(4):445-55. doi:10.1016/j.jse.2013.07.054]. Prior to the tensile test, a static preload of 5 N was applied to the sample for 5 seconds followed by 5 repeated loads from 5 to 50 N at a loading rate of 15 N/s. Thereafter, it was stretched at a speed of 1 mm/s until it failed. The load-to-failure ratio of the displacement was determined as a previously described value [Chung S W, Park H, Kwon J, Choe G Y, Kim S H, Oh J H. Effect of Hypercholesterolemia on Fatty Infiltration and Quality of Tendon-to-Bone Healing in a Rabbit Model of a Chronic Rotator Cuff Tear: Electrophysiological, Biomechanical, and Histological Analyses. Am J Sports Med 2016; 44(5):1153-64. doi:10.1177/0363546515627816]. The data from the tensile load-to-failure test was digitized and recorded using a personal computer-based data acquisition system. The entire procedure was performed by one orthopedic surgeon with the help of an expert with more than 10 years of experience in biomechanical experiments, ignorant of group allocation, and the results are shown in Table 4 and FIG. 7 below, and the tear patterns of each group are listed in Table 4.

TABLE 4

| | Group | Group | Group | Group | Group | |
|---|---|---|---|---|---|---|
| | A | B | C | D | E | P |
| | (n = 8) | (n = 8) | (n = 8) | (n = 8) | (n = 8) | value |
| Load-to-failure, N | 108.2 ± 3.6 | 111.6 ± 6.8 | 118.8 ± 9.1 | 122.4 ± 10.0 | 154.4 ± 18.0 | <0.001* |
| Tear mode (insertional | 6:2 | 5:3 | 4:4 | 5:3 | 2:6 | 0.339 |
| tear:mid-substance | | | | | | |
| tear), n | | | | | | |

N indicates Newtons, and n indicates number.
*Significantly different.

As shown in Table 4 and FIG. 7, after 12 weeks of suturing, the final load-to-failure of each group was as follows (mean±SD): group A: 108.2+3.6 N; group B: 111.6±6.8 N; group C: 118.8±9.1 N; group D: 122.4±10.0 N; and group E: 154.4±18.0 N; wherein group E showed the highest load-to-failure among all groups (P<0.001). Previously, it was shown that the mid-substance tear method was associated with better tendon to bone healing than the insertional tear method [Trudel G, Ramachandran N, Ryan S E, Rakhra K, Uhthoff H K. Supraspinatus tendon repair into a bony trough in the rabbit: mechanical restoration and correlative imaging. J Orthop Res 2010; 28(6):710-5. doi:10.1002/jor.21045]. The group A has 6 cases of insertional tear and 2 cases of mid-substance tear (25.0%), group B has 5 cases of insertional tear and 3 cases of mid-substance tear (37.5%), and group C has 4 cases of insertional tear and 4 cases (50.0%) of mid-substance tear, group D with 5 cases of insertional tear and 3 cases of mid-substance tear (37.5%), and group E with 2 cases of insertional tear and 6 cases of mid-substance tear (75.0%) (P=0.339).

This verified that the nanofiber sheet for healing of rotator cuff tears according to an aspect of the present invention has an excellent effect of healing a torn rotator cuff or a tear of a rotator cuff suture site or reducing re-tear after rotator cuff repair since it enhances the tendon to bone healing compared to direct administration of recombinant parathyroid hormone.

The nanofiber sheet according to an aspect of the present invention including the composition including recombinant parathyroid hormone has an excellent effect of healing of rotator cuff tears by improving tendon to bone healing when the sheet is locally fixed or attached to a suture site, specifically rotator cuff tear site or rotator cuff suture site, compared to direct systemic or local administration of recombinant parathyroid hormone.

### [Preparation Example 2] Preparation of Nanofiber Sheet 2 (rhPTH-Containing Nanofiber Sheet) For Healing of Rotator Cuff Tears

1. Fabrication of rhPTH Core-Shell Biocomposite Sheet

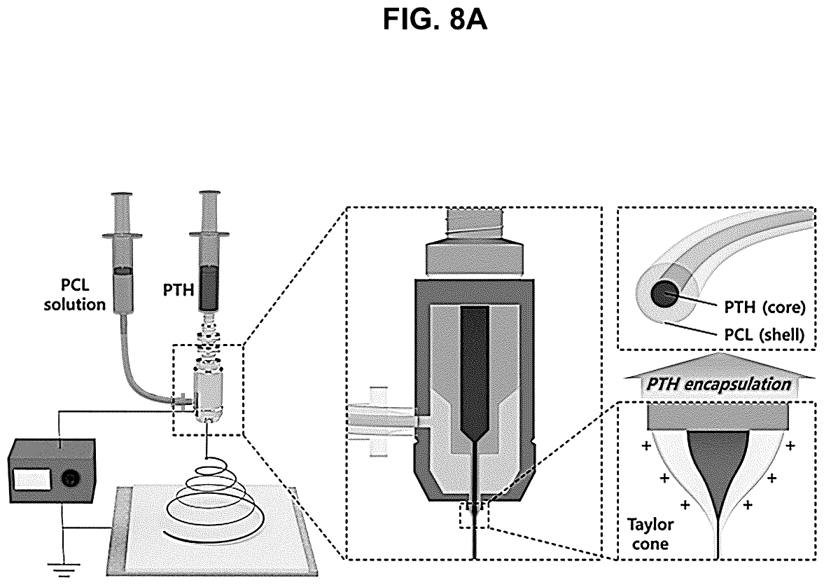

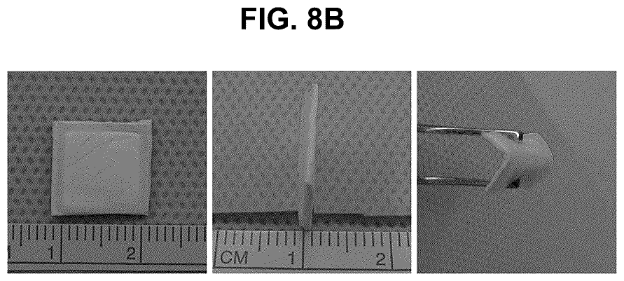

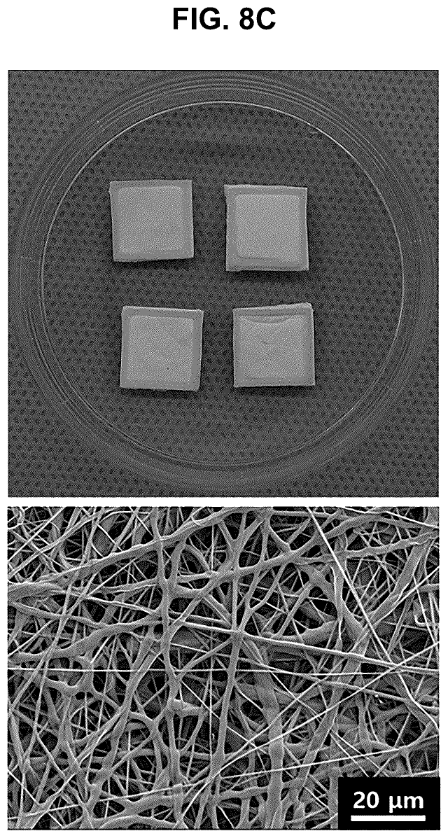

As shown in FIG. 8A, the rhPTH biocomposite was fabricated using coaxial electrospinning. A coaxial nozzle consisting of inner and outer nozzles with gauge sizes (hole diameter) of 27 G (0.2 mm) and 17 G (1.07 mm), respectively, was used. The rhPTH liquid and the PCL solution were injected into the inner and outer nozzles, respectively. The rhPTH liquid was prepared in the same way as the nanofiber sheet impregnated with rhPTH. The PCL solution was prepared by dissolving PCL at a concentration of 10 wt % in a mixture of DC and DMF in a volume ratio of 75:25. The infusion rates for the rhPTH liquid and the PCL solution were 0.5 mL/h and 7.0 mL/h, respectively. A voltage of 19 kV was applied to the nozzle tip 200 mm from the grounded collector. A microstructured framework was printed (FIG. 8B). The shape of nanofibers of the sheet was observed using scanning electron microscopy as shown in FIGS. 8C and 8D. In summary, rhPTH was made into a form in which nanofibers discharged from the external nozzle were packaged, and it was prepared as a paper-type support.

### [Example 2] Topical Attachment of Nanofiber Sheet for Healing of Rotator Cuff Tears

### [Example 2-1] Allocation of Rabbits

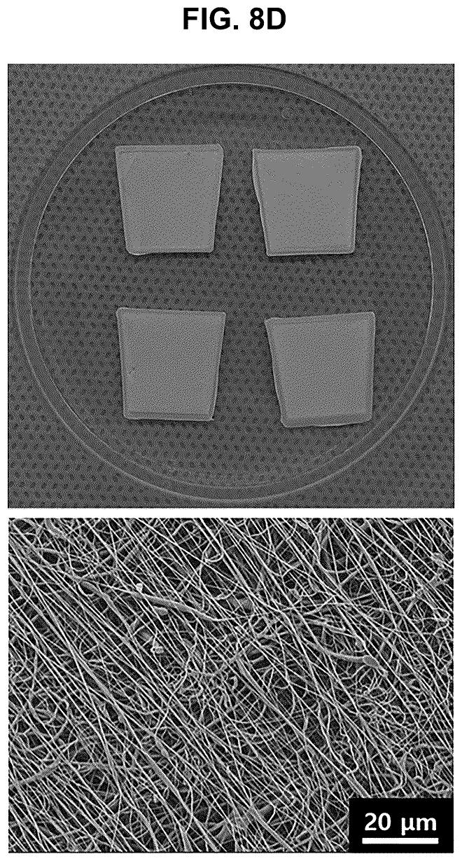

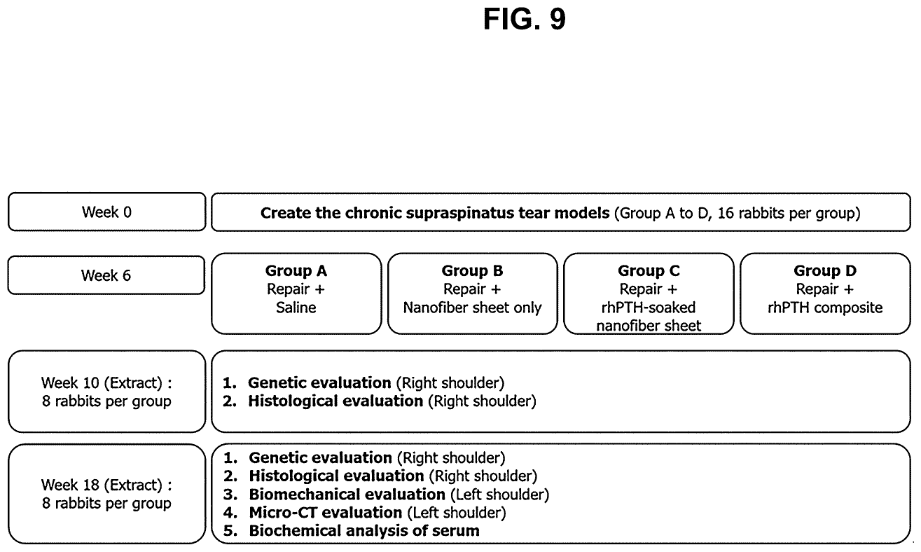

Sixty-four female New Zealand white rabbits (average weight: 3.0 to 3.5 kg, age: 6 months) were randomly allocated to 4 groups (n=16 each): group A (saline injection), group B (nanofiber sheet only), group C (rhPTH-soaked nanofiber sheet), and group D (rhPTH biocomposite). It was estimated that at least 6 sample sizes per group at each time point of euthanasia were required for sample size calculations (mean difference=15.2 N/kg, α error=0.05, β error=0.1) to detect significant differences in biomechanical data between groups. Given an expected dropout rate of 25%, at least 8 rabbits per group were required for each time point of euthanasia (FIG. 9).

### [Example 2-2] Surgical Procedure

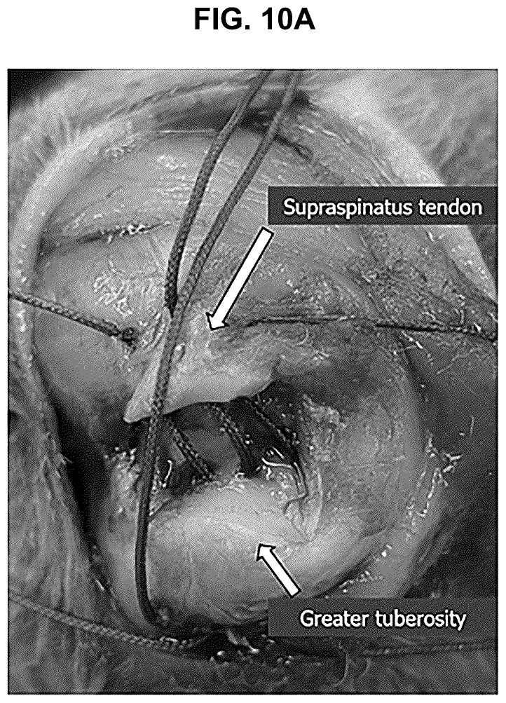

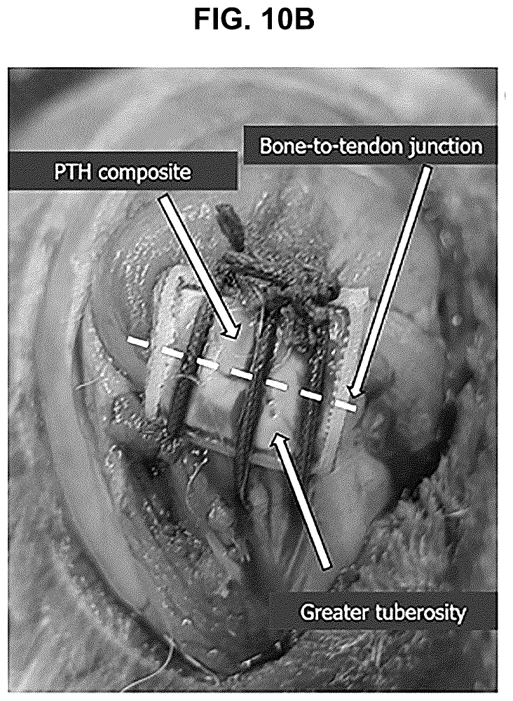

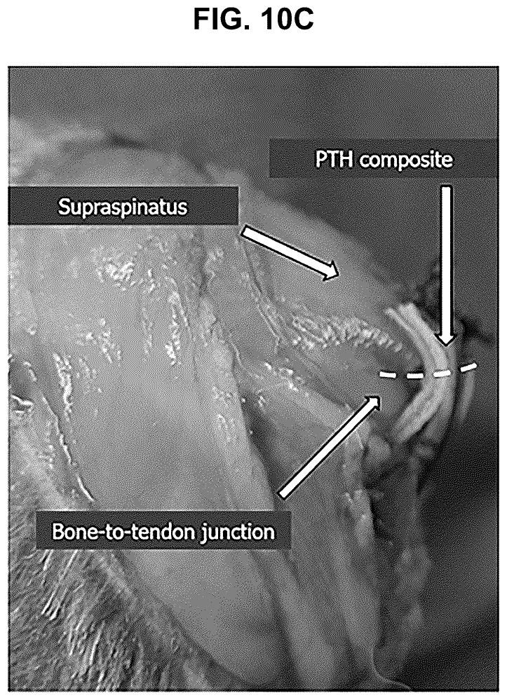

The 5 cm long incision was made bilaterally above the greater tuberosity of the proximal humerus. Using a scalpel blade, the supraspinatus tendon was sharply cut at the footprint of the greater tuberosity and wrapped with a Penrose drain (outer diameter of 8 mm, Yushin Co., Seoul, Korea). While the chronic RCT model was created according to the established protocol, the drainage tube was removed after 6 weeks of the supraspinatus tendon isolation and the chronic supraspinatus tendon tear condition was observed. Three 0.5 mm drill holes were made transversely through the proximal humerus in a posterior direction (FIG. 10A). The suture (2-0 Ticron; Tyco Healthcare, Mansfield) was passed through the prepared hole, and the supraspinatus tendon was reattached to the anatomical footprint in a tibial suture fashion. Before tightening the suture, the nanofiber sheet (groups B and C) and the biocomposite (group D) were implanted on the surface of the repair site (FIG. 10B). Thereafter, the suture was tied over the insert in a compression manner (FIG. 10C).

### [Experimental Example 2-1] In Vitro Test: rhPTH Release Kinetics

In vitro experiments on the rhPTH release kinetics were performed to confirm the sustained release capability of the rhPTH-soaked nanofiber sheet (nanofiber sheet 1) and the rhPTH biocomposite (nanofiber sheet 2) as previously described. Two different rhPTH scaffolds were incubated for 6 weeks at 4° C. using 50 mL phosphate buffered saline (PBS). Incubated for 1, 3, 6, 24, 72 hours and 1, 2, 3, 4, 5, 6 weeks, 100 μl liquid samples containing released rhPTH were taken from each culture medium and kept at −20° C. for 6 weeks. The amount of the released rhPTH was analyzed using the human PTH (1-34) ELISA (enzyme-linked immunosorbent assay) kit (EK-055-08, Phoenix, CA), and quantification was performed at an optical density of 450 nm using a microplate reader (ELx800, BioTek, Winooski, VT). All tests were performed in triplicate (each test value is indicated as 1^{st}, 2^{nd}, 3^{rd}) and the mean of the values for each time is listed in Table 5 (P value=0.031).

TABLE 5

| | rhPTH-soaked nanofiber sheet | | | | rhPTH biocomposite | | | |
|---|---|---|---|---|---|---|---|---|
| Time (hour) | 1st | 2nd | 3rd | Mean | 1st | 2nd | 3rd | Mean |
| 1 | 302.38 | 475.13 | 648.51 | 475.34 | 70.25 | 101.51 | 132.61 | 101.4566667 |
| 3 | 412.81 | 583.42 | 654.47 | 550.2333333 | 202.72 | 334.88 | 466.91 | 334.8366667 |
| 6 | 570.26 | 631.17 | 792.34 | 664.59 | 344.41 | 445.23 | 586.92 | 458.8533333 |
| 24 | 991.43 | 1285.42 | 1579.51 | 1285.453333 | 738.74 | 1089.61 | 1340.18 | 1056.176667 |
| 72 | 1387.19 | — | 1799.16 | 1593.66 | 1234.67 | — | 1416.59 | 1342.186667 |
| 168 | 1429.17 | — | 2071.48 | 1743.08 | 1489.18 | — | 1691.54 | 1573.76 |
| 336 | 1692.31 | — | 2041.32 | 1875.13 | 1754.39 | — | 2106.83 | 1932.116667 |
| 504 | 1694.56 | — | 2263.81 | 1984.38 | 2561.37 | — | 2923.53 | 2743.416667 |
| 672 | 1777.41 | — | 2374.19 | 2066.286667 | 3090.47 | — | 3592.56 | 3344.723333 |
| 840 | 1913.88 | — | 2361.29 | 2126.033333 | 3926.39 | — | 4352.48 | 4156.196667 |
| 1008 | 2087.17 | — | 2404.72 | 2261.113333 | 4275.91 | — | 5175.63 | 4725.743333 |

As a result, it was verified that not only the rhPTH-soaked nanofiber sheet (nanofiber sheet 1) has excellent rhPTH release ability, but also the rhPTH biocomposite (nanofiber sheet 2) has better rhPTH release ability than nanofiber sheet 1.

### [Experimental Example 2-2] Gene Expression Level Comparison Experiment After Attaching Nanofiber Sheet for Healing of Rotator Cuff Tears

To perform qRT-PCR, supraspinatus tendon tissues obtained from the right shoulder of rabbits (rabbits in each group of Example 2) were shredded with a masher at 4 and 12 weeks after suturing. Total RNA of the samples was then extracted using TRIzol reagent (Invitrogen, Waltham, MA, USA). For reverse transcription, 1 μg of total RNA and AMV First-Strand cDNA synthesis kit (Invitrogen, Waltham, MA) were used for cDNA synthesis. qRT-PCR was performed using the SYBR Green PCR MasterMix (Applicated Biosystems, Foster City, CA, USA) and the QuantStudio 6 Flex Real-Time PCR System (Applicated Biosystems). The PCR protocol used is as follows: repeating denaturation at 95° C. for 10 minutes, followed by 40 cycles of denaturation at 95° C. for 15 seconds and annealing at 58° C. for 60 seconds without extension to calculate values of the linear phase. The relative gene expression levels were calculated according to the 2-ACT formula using glyceraldehyde-3-phosphate dehydrogenase as a housekeeping gene. The primer sequences used in qRT-PCR are shown in Table 1 above.

The entire procedure was performed by an independent analyst ignorant of group allocation, and the results are shown in Table 6 below.

TABLE 6

| | After 4 weeks of suture | | | | | After 12 weeks of suture | | | | |
|---|---|---|---|---|---|---|---|---|---|---|
| | Group | Group | Group | Group | P | Group | Group | Group | Group | P |
| | A | B | C | D | Value | A | B | C | D | Value |
| COL1A1 | 0.99 | 1.05 | 1.41 | 1.97 | <0.001 | 0.83 | 0.97 | 1.09 | 1.16 | 0.112 |
| COL3A1 | 8.19 | 8.38 | 9.74 | 12.31 | <0.001 | 2.93 | 3.03 | 3.02 | 3.09 | 0.975 |
| SOX9 | 1.59 | 1.62 | 1.74 | 1.81 | 0.306 | 0.92 | 0.96 | 1.04 | 1.01 | 0.624 |
| BMP2 | 1.07 | 1.07 | 1.36 | 2.42 | 0.001 | 0.85 | 0.88 | 0.85 | 1.02 | 0.315 |
| SCX | 1.44 | 1.45 | 1.61 | 1.64 | 0.486 | 1.04 | 1.07 | 1.22 | 1.29 | 0.312 |
| ACAN | 1.23 | 1.26 | 1.31 | 1.31 | 0.701 | 1.49 | 1.51 | 1.44 | 1.54 | 0.767 |

It was found that the nanofiber sheet for healing of rotator cuff tears according to an aspect of the present invention increases the expression of COL1A1, COL3A1 related to collagen production, and BMP-2 related to bone formation, resulting in an excellent effect of healing torn rotator cuff.

### [Experimental Example 2-3] Histological Evaluation

The BTI tissue samples obtained from the right shoulder of each rabbit (rabbit in each group of Example 2) after 4 weeks and 12 of suturing were fixed overnight in neutral-buffered formalin, decalcified in hydrochloric acid (Leica Biosystems II, Surgipath Decalcifier, Wetzlar, Germany) for 8 to 12 hours, and then embedded in paraffin. The tissue sections were then deparaffinized and rehydrated in xylene to reduce alcohol concentration. The tissue sections of 5 μm thickness were stained with hematoxylin-eosin (HE) and Masson's trichrome according to the previously described protocol. The stained areas were observed with a Nikon Eclipse E200 microscope, and digital images were acquired using a Coolpix 995 camera (Nikon Japan, Tokyo). Safranin O staining was also performed to examine fibrocartilage regeneration in BTI using ImageJ software (National Institutes of Health, Bethesda, MD). The supraspinatus tendon insertion area was manually outlined and measured using ImageJ software. The relative proportion of red-positive staining area to the summarized supraspinatus tendon insertion area was calculated to evaluate the regenerative fibrocartilage area. All evaluations were performed by two experienced investigators who were randomly blinded under the guidance of an experienced pathologist, and were conducted according to the semi-quantitative grading system (0 to 3 grades; the grading of continuity, density, and orientation of collagen fibers is the same as the grading of continuity, density, and orientation of collagen fibers in Experimental Example 1-2; the vascularity and cellularity of collagen fibers were graded on a scale of 0 to 3, corresponding to none or minimal, mild, moderate, and marked, respectively) and the mean value adoption, and the results are summarized in Table 7.

TABLE 7

| | Group | | | | Group | | | | Group | | | | Group | | | | |
|---|---|---|---|---|---|---|---|---|---|---|---|---|---|---|---|---|---|
| | A(n = 8) | | | | B(n = 8) | | | | C(n = 8) | | | | D(n = 8) | | | | P |
| Parameter | G0 | G1 | G2 | G3 | G0 | G1 | G2 | G3 | G0 | G1 | G2 | G3 | G0 | G1 | G2 | G3 | value |

After 4 weeks of suture

| | | | | | | | | | | | | | | | | | | |
|---|---|---|---|---|---|---|---|---|---|---|---|---|---|---|---|---|---|---|
| Collagen | Vascularity | 0 | 5 | 2 | 1 | 0 | 4 | 3 | 1 | 0 | 1 | 2 | 5 | 0 | 0 | 3 | 5 | 0.001 |
| Fibers | Cellularity | 0 | 6 | 1 | 1 | 0 | 5 | 3 | 0 | 0 | 1 | 2 | 5 | 0 | 1 | 1 | 6 | 0.001 |
| | Continuity | 6 | 2 | 0 | 0 | 6 | 1 | 1 | 0 | 5 | 2 | 1 | 0 | 4 | 3 | 1 | 0 | 0.237 |
| | Density | 6 | 1 | 1 | 0 | 5 | 2 | 1 | 0 | 5 | 1 | 2 | 0 | 4 | 2 | 2 | 0 | 0.323 |
| | Orientation | 7 | 1 | 0 | 0 | 6 | 2 | 0 | 0 | 6 | 1 | 1 | 0 | 5 | 2 | 1 | 0 | 0.182 |

| | | | | | | | | | | | | | | | | | |
|---|---|---|---|---|---|---|---|---|---|---|---|---|---|---|---|---|---|
| TTB junction | 5 | 3 | 0 | 0 | 5 | 2 | 1 | 0 | 4 | 3 | 1 | 0 | 4 | 2 | 2 | 0 | 0.269 |
| maturation | | | | | | | | | | | | | | | | | |

After 12 weeks of suture

| | | | | | | | | | | | | | | | | | | |
|---|---|---|---|---|---|---|---|---|---|---|---|---|---|---|---|---|---|---|
| Collagen | Vascularity | 4 | 4 | 0 | 0 | 5 | 2 | 1 | 0 | 5 | 3 | 0 | 0 | 6 | 2 | 0 | 0 | 0.323 |
| Fibers | Cellularity | 5 | 3 | 0 | 0 | 6 | 1 | 1 | 0 | 6 | 2 | 0 | 0 | 7 | 1 | 0 | 0 | 0.291 |
| | Continuity | 5 | 3 | 0 | 0 | 4 | 3 | 1 | 0 | 1 | 2 | 1 | 4 | 0 | 0 | 0 | 8 | <0.001 |
| | Density | 6 | 2 | 0 | 0 | 5 | 3 | 0 | 0 | 2 | 1 | 1 | 4 | 0 | 0 | 0 | 8 | <0.001 |
| | Orientation | 0 | 3 | 2 | 3 | 0 | 3 | 1 | 4 | 0 | 2 | 2 | 4 | 0 | 0 | 0 | 8 | 0.022 |

| | | | | | | | | | | | | | | | | | |
|---|---|---|---|---|---|---|---|---|---|---|---|---|---|---|---|---|---|
| TTB junction | 4 | 4 | 0 | 0 | 3 | 5 | 0 | 0 | 2 | 1 | 1 | 4 | 0 | 0 | 0 | 8 | 0.269 |
| maturation | | | | | | | | | | | | | | | | | |

It was found that compared to direct administration of recombinant parathyroid hormone, the nanofiber sheet for healing of rotator cuff tears according to an aspect of the present invention has an excellent effect of healing a torn rotator cuff or a tear of a rotator cuff suture site or reducing re-tear after rotator cuff repair since it enhances at least one selected from the group consisting of the vascularity, the cellularity, the continuity, the density, the orientation of collagen fibers, and the maturation in tendon to bone junctions (TTB junction maturation).

### [Experimental Example 2-4] Biomechanical Evaluation

The entire tendon to bone junction unit from the left shoulder of each rabbit (rabbit in each group of Example 2) after 12 weeks of suturing was used to evaluate the biomechanical properties of the repaired rotator cuff with a universal material testing machine (AGS-X; Shimadzu, Kyoto, Japan) as previously reported. Prior to the tensile test to measure the load-to-failure, a static preload of 5 N was applied to the sample for 5 seconds, followed by 5 repeated loads from 5 to 50 N at a loading rate of 15 N/s. Thereafter, it was stretched at a speed of 1 mm/s until it failed. During testing, samples were kept moist with 0.9% saline to mimic the internal environment. The test results are shown in Table 8.

TABLE 8

| | Group | Group | Group | Group | P |
|---|---|---|---|---|---|
| Mean | A(n = 8) | B(n = 8) | C(n = 8) | D(n = 8) | value |
| Load-to | 28.9 | 30.1 | 39.7 | 48.2 | <0.001 |
| Failure(N/kg) | | | | | |
| Stiffness | 16.1 | 16.3 | 17.1 | 19.1 | 0.297 |

This verified that the nanofiber sheet for healing of rotator cuff tears according to an aspect of the present invention has an excellent effect of healing a torn rotator cuff or a tear of a rotator cuff suture site or reducing re-tear after rotator cuff repair since it enhances the tendon to bone healing compared to direct administration of recombinant parathyroid hormone.

### [Experimental Example 2-5] Micro-CT Analysis: Healing Quality of BTI Bone Marrow

Micro-CT analysis was performed on the remaining proximal humerus after mechanical testing. After removing the remaining soft tissue, the humerus samples (humeral bone samples of rabbits in each group of Example 2) were fixed using a solution of ethanol and sterile water in a ratio of 1:1 for 24 hours at room temperature. The following parameters were measured using a desktop micro-CT system (Skyscan 1076, Skyscan, Belgium): bone mineral density (BMD), tissue mineral density (TMD), and bone volume fraction (bone volume/total volume (BV/TV)) of the supraspinatus tendon footprint. The Micro-CT was performed at 40 kV and 170 mA with the integration time of 300 ms and the resolution of 18.22 μm according to the established protocol. On the surface of the supraspinatus tendon repair site, a user-defined cylindrical region of interest with a diameter of 10 mm was created after acquiring 3D reconstruction images. Thereafter, an orthopedic surgeon who was not participating in the study evaluated the relevant parameters using ImageJ software, which is shown in Table 9.

TABLE 9

| | Group | Group | Group | Group | P |
|---|---|---|---|---|---|
| Mean | A(n = 8) | B(n = 8) | C(n = 8) | D(n = 8) | value |
| BMD | 393.8 | 402.7 | 449.5 | 513.9 | <0.001 |
| (mg/mm³) | | | | | |
| TMD | 460.8 | 474.2 | 557.9 | 627.2 | <0.001 |
| (mg/mm³) | | | | | |
| BV/TV (%) | 61.8 | 63.3 | 71.3 | 80.4 | <0.001 |

It was found that the nanofiber sheet for healing of rotator cuff tears according to an aspect of the present invention has an excellent effect of increasing bone mineral density and tissue mineral density, and has excellent healing ability of the bone layer of the humerus by increasing the bone volume fraction of the supraspinatus tendon footprint.

### [Experimental Example 2-6] Serum Chemistry Analysis

As previously described, assays were performed using the quantitative sandwich enzyme immunoassay technique as previously described. The blood samples (blood samples from rabbits in each group of Example 2) were collected by cardiac puncture using a blood collection tube after 12 weeks of suturing. The sample was immediately centrifuged at 3000 rpm for 10 minutes, and the separated serum was collected and stored at −80° C. until ELISA analysis was performed. The liquid was diluted 50-fold before analysis. The OCN ELISA kit (RK03431, ABclon, Woburn, MA), PINP ELISA kit (MBS017936, San Diego, CA MyBio Source, CA) and AP (APXA, Cambridge, ABXA Kit) were used to measure the serum levels of osteocalcin (OCN), procollagen type IN-terminal propeptide (PINP), and alkaline phosphatase (AP). The results are shown in Table 10.

TABLE 10

| | Group | Group | Group | Group | P |
|---|---|---|---|---|---|
| Mean | A(n = 8) | B(n = 8) | C(n = 8) | D(n = 8) | value |
| Osteocalcin | 7.745 | 7.81875 | 8.11875 | 8.4275 | 0.522 |
| (ng/mL) | | | | | |
| PINP (ng/mL) | 1.11125 | 1.1325 | 2.3475 | 3.34875 | <0.001 |
| alkaline | 2.6125 | 2.7875 | 4.47 | 6.72875 | <0.001 |
| phosphatase | | | | | |
| (mIU/mL) | | | | | |

It was found that the nanofiber sheet for healing of rotator cuff tears according to an aspect of the present invention has an excellent effect of significantly increasing the expression of OCN, PINP, and AP, healing a torn rotator cuff or a tear of a rotator cuff suture site or reducing re-tear after rotator cuff repair.

In summary, both nanofiber sheet 1 and nanofiber sheet 2 demonstrated superior rotator cuff healing effects compared to other control groups in animal experiments using rabbits. In the animal experiment study, the nanofiber sheet was partially decomposed at 4 weeks after insertion, but was completely degraded when confirmed at 12 weeks. It was found that, on the genetic analysis performed at the 4^{th} week after nanofiber sheet insertion, the expression of COL1A1 involved in collagen production of the nanofiber sheet 1 and the expression of COL1A1, COL3A1 involved in collagen production, and BMP-2 involved in bone formation of the nanofiber sheet 2 were higher than those of other control groups.

On histological analysis performed at 12 weeks after nanofiber sheet insertion, it was confirmed that both nanofiber sheets 1 and 2 had the higher density, continuity, orientation, and recovery maturation of collagen compared to the other control groups, and had the stronger tensile strength than the other control groups even in biomechanical analysis in which the mechanical tensile strength was tested.

Considering the above experimental results, the nanofiber sheet of the present invention is not only suitable for the purpose of locally releasing rhPTH as it remains in the body without decomposition during the period during which the repair of the suture is accelerated, but also has the high biological stability since it is completely degraded at 12 weeks of suture and increases the expression of genes involved in suture recovery, resulting in promoting histological recovery.

What is claimed is:

1. A nanofiber sheet for healing of a rotator cuff tear, the nanofiber sheet comprising:
a composition comprising teriparatide, isomers thereof, pharmaceutically acceptable salts thereof, hydrates thereof, or solvates thereof as an active ingredient.

2. The nanofiber sheet of claim 1,
wherein the sheet is impregnated with the composition.

3. The nanofiber sheet of claim 1,
wherein nanofibers constituting the nanofiber sheet contain the composition therein.

4. The nanofiber sheet of claim 3,
wherein the nanofibers have a core-shell structure in which a nanofiber shell surrounds a core comprising the composition.

5. The nanofiber sheet of claim 1,
wherein the rotator cuff tear is a chronic or degenerative rotator cuff tear.

6. The nanofiber sheet of claim 1,
wherein the healing of the rotator cuff tear includes healing of tear at a suture site after rotator cuff repair.

7. The nanofiber sheet of claim 1,
wherein the sheet is locally attached to the rotator cuff tear site.

8. The nanofiber sheet of claim 1,
wherein the sheet enhances at least one selected from the group consisting of vascular permeability, cellular density, continuity, density, orientation of collagen fibers of rotator cuff, and maturation of tendon to bone junctions.

9. The nanofiber sheet of claim 1,
wherein the sheet increases the expression level of one or more genes, compared to the sheet unattached group or before sheet attachment, selected from the group consisting of collagen type I alpha 1 (COL1A1), collagen type Ill alpha 1 (COL3A1), bone morphogenetic protein 2 (BMP-2), scleraxis (SCX), SRY-box 9 (SOX9) and aggrecan (ACAN).

10. The nanofiber sheet of claim 1,
wherein the sheet is an electrospun nanofiber sheet.

11. The nanofiber sheet of claim 1,
wherein the composition further comprises hyaluronic acid or salts thereof.

12. A method of manufacturing a nanofiber sheet for healing of a rotator cuff tear, the method comprising:
incorporating a composition comprising teriparatide, isomers thereof, pharmaceutically acceptable salts thereof, hydrates thereof, or solvates thereof as an active ingredient in the formed sheet.

13. The method of claim 12,
forming the nanofiber sheet by electrospinning an electrospinning solution; and
impregnating the nanofiber sheet with a composition comprising teriparatide, isomers thereof, pharmaceutically acceptable salts thereof, hydrates thereof, or solvates thereof as an active ingredient.

14. The method of claim 13,
wherein the electrospinning solution is produced by adding, to an organic solvent, at least one selected from the group consisting of polycaprolactone (PCL), collagen, gelatin, elastin, chitosan, silk fibroin, alginate, poly(glycolic acid) (PGA), poly(lactic-co-glycolic acid) (PLGA), poly(L-lactic acid) (PLLA), poly(L-lactic acid)/collagen (PLLA/CL), polyvinyl alcohol (PVA), and polyethylene oxide (PEO).

15. The method of claim 13,
wherein the composition further comprises hyaluronic acid or salts thereof.

16. The method of claim 12, further comprising:
forming nanofibers containing the composition therein by discharging the electrospinning solution from an external nozzle, and by discharging the composition containing teriparatide, isomers thereof, pharmaceutically acceptable salts thereof, hydrates thereof, or solvates thereof as an active ingredient from an internal nozzle.

17. The method of claim 16,
wherein the electrospinning solution is produced by adding, to an organic solvent, at least one selected from the group consisting of polycaprolactone (PCL), collagen, gelatin, elastin, chitosan, silk fibroin, alginate, poly(glycolic acid) (PGA), poly(lactic-co-glycolic acid) (PLGA), poly(L-lactic acid) (PLLA), poly(L-lactic acid)/collagen (PLLA/CL), polyvinyl alcohol (PVA), and polyethylene oxide (PEO).


SEQUENCE LISTING (14 sequences)

SEQ ID NO: 1 - DNA, 20 bp, synthetic construct
  source 1..20: mol_type = other DNA
ggtcttctgcgacatggaca

SEQ ID NO: 2 - DNA, 20 bp, synthetic construct
  source 1..20: mol_type = other DNA
ccacacgtgcttcttctcct

SEQ ID NO: 3 - DNA, 20 bp, synthetic construct
  source 1..20: mol_type = other DNA
gctctgcttcatcccactgt

SEQ ID NO: 4 - DNA, 20 bp, synthetic construct
  source 1..20: mol_type = other DNA
atatttggcacggttcgggt

SEQ ID NO: 5 - DNA, 20 bp, synthetic construct
  source 1..20: mol_type = other DNA
gggtggaacgactggattgt

SEQ ID NO: 6 - DNA, 20 bp, synthetic construct
  source 1..20: mol_type = other DNA
tgcacgatggcatggttagt

SEQ ID NO: 7 - DNA, 20 bp, synthetic construct
  source 1..20: mol_type = other DNA
acagatctgcaccttctgcc

SEQ ID NO: 8 - DNA, 20 bp, synthetic construct
  source 1..20: mol_type = other DNA
ccgtgactcttcagtggcat

SEQ ID NO: 9 - DNA, 20 bp, synthetic construct
  source 1..20: mol_type = other DNA
gcccagaagagcctcaaagt

SEQ ID NO: 10 - DNA, 20 bp, synthetic construct
  source 1..20: mol_type = other DNA
ggtaccagttgccttcagct

SEQ ID NO: 11 - DNA, 20 bp, synthetic construct
  source 1..20: mol_type = other DNA
ggatctaccgctgtgaggtg

SEQ ID NO: 12 - DNA, 20 bp, synthetic construct
  source 1..20: mol_type = other DNA
gtggagatggcccgatagtg

SEQ ID NO: 13 - DNA, 20 bp, synthetic construct
  source 1..20: mol_type = other DNA
ggaatccactggcgtcttca

SEQ ID NO: 14 - DNA, 20 bp, synthetic construct
  source 1..20: mol_type = other DNA
ggttcacgcccatcacaaac